(19) United States
(12) Patent Application Publication
(10) Pub. No.: US 2024/0293400 A1
(43) Pub. Date: Sep. 5, 2024

(54) NOVEL AZAINDOLE DERIVATIVES AS ANTIVIRAL AGENTS

(71) Applicant: Centre National de la Recherche Scientifique, Paris (FR)

(72) Inventors: Laurence BRIANT, GALLARGUES-LE-MONTUEUX (FR); Eric BERNARD, MONTPELLIER (FR); Camille CLOP, FOURNES (FR)

(21) Appl. No.: 18/563,260

(22) PCT Filed: May 23, 2022
(86) PCT No.: PCT/FR2022/050979

§ 371 Date: Nov. 21, 2023

(30) Foreign Application Priority Data
May 21, 2021 (FR) FR2105359

(51) Int. Cl. A61K 31/496; A61K 45/06; A61P 31/14

(52) U.S. Cl. CPC A61K 31/496; A61K 45/06; A61P 31/14

(57) ABSTRACT

The present invention relates to compounds derived from 7-azaindole useful as inhibitors of AXL kinases for the treatment of viral infections. In particular, the invention relates to compounds of the following formula (I): (I) for their use in the prevention and/or treatment of viral infections. The present invention also relates to their method of preparation.

### FIELD OF THE INVENTION

The present invention relates to compounds derived from 7-azaindole useful as inhibitors of AXL kinases for the treatment of viral infections. The present invention also relates to their method of preparation.

### TECHNICAL BACKGROUND

AXL is a Receptor Tyrosine Kinase (RTK) belonging to the TAM family composed of TYRO-3, AXL and MER. Initially discovered and demonstrated in patients suffering from chronic myeloid leukemia (CML), the involvement of the AXL receptor in viral infections such as, for example, dengue, the Zika virus, Chikungunya or Ebola fever was described in the literature (Chen, J. et al. Nature Microbiology, 2018, 3, pp.302-309; Fedeli, C. et al. Journal of Virology, 2018, 92:e01613-17; Hastings, A. K. et al., iScience, 2019, 13 pp.339-350; Meertens, L. et al., Cell Reports, 2017, 18, 3, pp.324-333). This receptor, by interacting via the Gas6 adapter with phosphatidylserines present in the viral envelopes, promotes the attachment of the enveloped viruses to their cell target. AXL thus stimulates viral endocytosis. The engagement of AXL during this interaction stimulates the phosphorylation of the AXL intracytoplasmic domain and activates the associated signaling pathways. These signals neutralize the production of interferons and the associated antiviral responses, promoting the escape of these viruses from the immune control of the host.

To date, there are several active kinase inhibitors on AXL (Myer et al., J. Med. Chem. (2015), 59(8): 3593-3608) but none is really selective or specific for this kinase. In a large majority of cases, the activity on AXL is secondary with respect to the desired main activity on MET or MER due to the similarity of sequence between these RTKs. It is in particular the case for Bosutinib or Cabozantinib, which are multi-targeted kinase inhibitors or MTKIs already on the market, or BMS777607 (currently in clinical phase 2). This is also the case of the 7-azaindole derivative NPS-1034, which has a relatively broad inhibition profile, by inhibiting a large panel of kinases such as AXL/DDR1/FLT3/KIT/MEK/MET/ROS1 and TIE1.

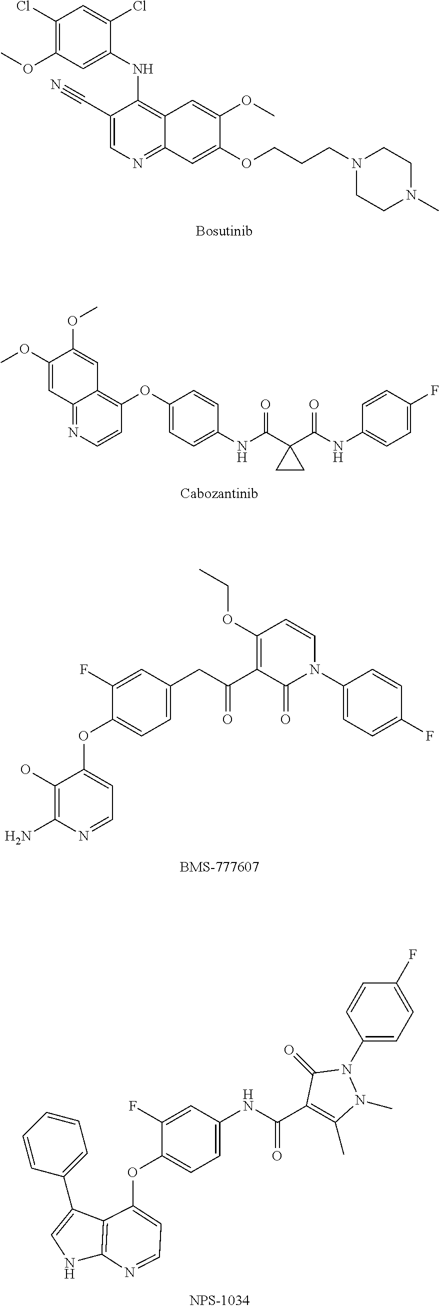

Another AXL kinase inhibitor is Bemcentinib (also called R428 or BGB324), currently in the clinical phase. This compound—which has a very different structure from kinase inhibitors currently on the market—has also been revealed to be active on other kinases such as ABL/KIT/JAK2-3/LCK/PDGFRB/TIE2.

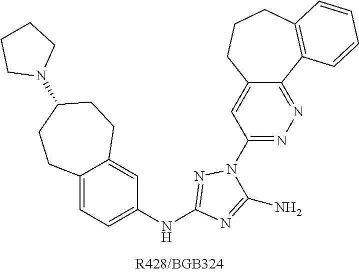 [SMILES: Nc1nc(Nc2ccc3c(c2)CC[C@@H](N2CCCC2)CC3)nn1-c1cc2c(nn1)-c1ccccc1CCC2]

There is therefore a need for novel AXL inhibitor compounds which are more selective and effective with respect to viral infections.

The present invention thus proposes novel 7-azaindole derivatives that strongly inhibit AXL kinase for use as antiviral agents.

By virtue of this very specific and unique inhibition profile for this type of structure, these compounds can be used in therapy in the treatment of infections caused by viruses using the AXL receptor to multiply. In order to combat viral infections, by inhibiting the phosphorylation of AXL, these compounds will act both by blocking the endocytosis of the enveloped viruses in their cell target, and by neutralizing the intracellular signals controlled by AXL which are known to inhibit the production of the interferons and of the antiviral responses of the host. The inhibitors of the present invention can thus be used for the treatment of diseases in which AXL are involved, in particular viral infections and in particular viral input in the cells.

### SUMMARY OF THE INVENTION

The present invention thus relates to a compound of formula (I):

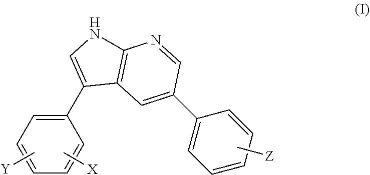

in which
X represents a hydrogen atom or a halogen atom,
Y is selected from a hydrogen atom, a —CH₂OH, —CH═NOH, —CO₂H group, an aryl optionally mono- or polysubstituted, a heteroaryl optionally mono- or polysubstituted, and -L-(CH₂)p-W,
   L represents —C(O)NH—, —CH₂NH—, —NHC(O)—, —CH₂N═CH—, —CH═N—, —NHC(O)NH— or —CH₂NHC(O)—,
   p is an integer from 0 to 2,
   when L represents —CH₂NH—, —NHC(O)—, —CH₂N═CH—, —CH═N—, —NHC(O)NH— or —CH₂NHC(O)—, W is selected from:
   a hydrogen atom,
   a C₁-C₆ alkyl, preferably a methyl,
   a C₆-C₁₀ aryl, optionally mono- or polysubstituted, advantageously by 1 to 3 groups independently selected from a halogen atom, a hydroxyl, a C₁-C₆ alkyl, or a C₁-C₆ alkoxyl, and
   a 5- to 10-membered heteroaryl containing from 1 to 3 heteroatoms independently selected from N, O and S, said heteroaryl optionally being mono- or polysubstituted, advantageously it is optionally substituted with 1 to 4 groups independently selected from:
   a halogen atom,
   a hydroxyl,
   an oxo group
   a C₁-C₆ alkoxyl,
   a C₁-C₆ alkyl, wherein 1 or several hydrogen atoms is optionally replaced with a fluorine atom, and
   a C₆-C₁₀ aryl, optionally substituted with a halogen atom and/or a C₁-C₆ alkyl;
   when L represents —C(O)NH—, W is selected from:
   an optionally mono- or polysubstituted phenyl, advantageously it is optionally substituted with a halogen atom, a hydroxyl group, a C₁-C₆ alkyl, or a C₁-C₆ alkoxyl group, and
   a 5- to 10-membered heteroaryl comprising from 1 to 2 heteroatoms independently selected from N, O and S, said heteroaryl optionally being mono- or polysubstituted, advantageously it is optionally substituted with 1 to 4 substituents independently selected from:
   a halogen atom,
   a hydroxyl,
   an oxo group,
   a C₁-C₆ alkoxyl group,
   a C₁-C₆ alkyl, wherein 1 or several hydrogen atoms is optionally replaced with a fluorine atom, and
   a C₆-C₁₀ aryl, optionally substituted with a halogen atom and/or a C₁-C₆ alkyl;
Z represents —CH₂Q or —O(CH₂)ₘT,
   Q represents —OH, —SO₂R₁, —NR₂R₃, or —R₄
   R₁ represents a C₁-C₆ alkyl,
   R₂ and R₃ each independently represent a C₁-C₆ alkyl, and
   R₄ represents a 5-7 membered heterocycloalkyl, comprising 1 to 2 heteroatoms independently selected from N, O and S, said heterocycloalkyl optionally substituted with 1 to 3 substituents independently selected from a C₁-C₆ alkyl, a C₁-C₆ C(O)-alkyl and a C₁-C₆ C(O)O-alkyl,
   m is equal to 1 or 2,
   T is —O(CH₂)ₙCH₃ or —R₅
   n is 0 or 1
   R₅ is a 5-7 membered heterocycloalkyl, comprising 1 to 2 heteroatoms independently selected from N, O and S, said heterocycloalkyl optionally substituted with 1 to 3 substituents independently selected from a C₁-C₆ alkyl, a C₁-C₆ C(O)-alkyl and a C₁-C₆ C(O)O-alkyl,
a pharmaceutically acceptable salt thereof or a mixture thereof,
for use in the prevention and/or treatment of viral infections.

The present invention also relates to a pharmaceutical composition comprising a compound according to the invention or a pharmaceutically acceptable salt thereof as an active ingredient, and a pharmaceutically acceptable excipient, for use in the prevention or treatment of viral infections.

### DETAILED DESCRIPTION

As a general rule, the following terms and definitions are used.

The expression “peptide coupling” in the present invention refers to the reaction for forming an amide-NH—C(O)-bond. The techniques used in this reaction are common to peptide syntheses, that is to say, by activation of a carboxylic acid to react with an amine. The peptide coupling reactions used in the present invention are thus derived from peptide syntheses, and directly applicable to the subject matter of the present invention.

The peptide coupling reactions are well known to a person skilled in the art, and can in particular be carried out using a coupling agent such as, N,N′-dicyclohexylcarbodiimide (DCC) or 1-ethyl-3-(3′-dimethylaminopropyl)carbodiimide hydrochloride (EDC), or N-hydroxy-5-norbornene-2,3-dicarbodiimide), or a benzotriazole (such as O-(1H-benzotriazol-1-yl)-N,N,N′,N′-tetramethyluronium (TBTU), benzotriazol-1-yl-oxytris(dimethylamino)phosphonium hexafluorophosphate (BOP), 0-(7-azabenzotriazol-1-yl)-1,2,3-tetramethyluronium hexafluorophosphate (HATU), O-benzotriazol-1-yl-N,N,N′,N′-tetramethyluronium hexafluorophosphate (HBTU), O-benzotriazol-1-yl-tetramethyl tetrafluoroborate (TBTU)), or an N-hydroxybenzotriazole (HOBT)/EDCI mixture, in a solvent such as chloroform, dichloromethane, dichloroethane, ethyl acetate, dimethylformamide (DMF), tetrahydrofuran (THF), dimethylsulfoxide (DMSO), N-methyl pyrrolidinone (NMP), preferably at a temperature of between 20° C. and 150° C.

Alternatively, peptide coupling takes place by first activating the carboxylic acid by transformation into acyl chloride (in particular in the presence of thionyl chloride or acetyl chloride) or a corresponding anhydride (for example in the presence of acetic anhydride or isopropyl), then reaction with the desired amine, preferably in the presence of a base to neutralize the acid released during the reaction (in particular HCl in the case of an acyl chloride).

The term C(O) is equivalent to “C═O”.

The expression “alkyl” or “alkyl group” in the present invention denotes a saturated linear or branched aliphatic group containing 1 to 6 carbon atoms, if not otherwise defined. Examples of alkyl groups covered by the subject matter of the present invention are methyl, ethyl, propyl, butyl, tert-butyl, isopropyl groups.

In some embodiments, it is specified that one or more hydrogen atoms of the alkyl group are optionally replaced by a fluorine atom. In this case, preferably, 1 to 3 hydrogen atoms at most are affected. An example is the group CH₂CF₃.

The expression “aryl group” or “aryl” in the present invention denotes a (mono- or polycyclic) cyclic aromatic group containing between 6 and 10 carbon atoms. Examples of aryl groups covered by the subject matter of the present invention are the phenyl, napthyl, preferably phenyl groups.

The expression “heteroaryl group” or “heteroaryl” in the present invention denotes a 5- to 10-membered (mono- or polycyclic) aromatic cyclic group containing between 2 and 9 carbon atoms and between 1 and 3 heteroatoms independently selected from nitrogen, oxygen or sulfur. Examples of heteroaryl groups are the furan, pyrrole, thiophene, thiazole, isothiazole, imidazole, oxazole, isoxazole, pyrazole, pyridine, pyrazine, pyridazine, pyrimidine, quinoline, indole, quinoxaline, benzofuran, dihydrobenzofuran, benzodioxole, benzotriazole, benzimidazole groups, preferably chosen from pyrrole, imidazole, thiophene, indole, pyrimidine, benzofuran, benzodioxole, indole and benzimidazole.

The expression “heterocycloalkyl” or “heterocycloalkyl group” in the present invention denotes a 5- to 7-membered cyclic aliphatic group, comprising one or more (preferably 1 to 2) heteroatoms independently selected from nitrogen, oxygen or sulfur, such as morpholine, piperidine, piperazine, pyrrolidine, homopiperazine (1,4-diazacycloheptane). Preferably, it is morpholine or piperazine.

The expression “halogen atom” in the present invention denotes a fluorine, chlorine, bromine or iodine atom. Preferably, it is chlorine or fluorine, in particular fluorine.

The expression “alkoxyl group” in the present invention refers to an oxygen-bonded alkyl group. Examples of alkoxyl groups are methoxy, ethoxy, propoxy, isopropoxy, butoxy, isobutoxy, tert-butoxy groups. Preferably, it is a methoxy or ethoxy group.

The expression “aryloxy group” in the present invention denotes an aryl group bonded to an oxygen atom. Examples of aryloxy groups are phenyloxy groups.

The expression “hydroxyl group” or “hydroxyl” in the present invention refers to: OH.

The expression “oxo group” denotes the substituent: ═O.

The expression “optionally substituted aryl or heteroaryl group” in the present invention refers to an aryl or heteroaryl optionally substituted by one or more (preferably 1 to 3, even more preferably 1 or 2) substituents independently selected from: a halogen atom, a nitro group —(NO₂), a cyano (CN) group, a C₁-C₆ alkoxyl group, a C₅-C₁₀ aryloxy group, a C₁-C₆ alkyl group wherein 1 or more hydrogen atoms is optionally replaced with a fluorine atom, a heteroaryl group, a hydroxyl group, an oxo group, a C₁-C₆ -CONH-alkyl group, a C₁-C₆ -NHCOalkyl group, and a NR₂R₃ group wherein R₂ and R₃ independently represent a C₁-C₆ alkyl group, or a C₆-C₁₀ aryl group optionally substituted with a halogen atom and/or a C₁-C₆ alkyl group.

Preferably, the substituents in the expression “optionally substituted aryl or heteroaryl group” are independently selected from:
a halogen atom, in particular a fluorine or a chlorine,
a hydroxyl,
an oxo group,
a C₁-C₆ alkoxyl, in particular a methoxy,
a C₁-C₆ alkyl, wherein one or more hydrogen atoms is/are optionally replaced with a fluorine atom, preferably a methyl group or CH₂CF_{3,} and
a C₆-C₁₀ aryl group optionally substituted with a halogen atom (in particular a fluorine or a chlorine) and/or a C₁-C₆ alkyl group, for example a fluorophenyl (preferably 4-fluorophenyl) or a methylfluorophenyl (for example 4-fluoro-2-methylphenyl);

The expression “7-azaindole” in the present invention refers to 1H-pyrrolo [2,3-b]pyridine:

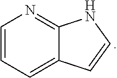

In the present invention, “pharmaceutically acceptable” refers to what is useful in the preparation of a pharmaceutical composition which is generally safe, non-toxic and neither biologically nor otherwise undesirable and which is acceptable for veterinary use as well as human pharmaceutical use.

In the present invention, the expression “pharmaceutical composition” refers to any composition consisting of an effective dose of at least one compound of the invention and at least one pharmaceutically acceptable excipient. Such excipients are selected, depending on the pharmaceutical form and on the desired method of administration, from excipients usually known by a person skilled in the art.

“Pharmaceutically acceptable salts” of a compound refers to salts which are pharmaceutically acceptable, as defined herein, and which have the desired pharmacological activity of the parent compound. Such salts comprise:
(1) hydrates and solvates,
(2) pharmaceutically acceptable acid addition salts formed with pharmaceutically acceptable inorganic acids such as hydrochloric acid, hydrobromic acid, sulfuric acid, nitric acid, phosphoric acid and the like; or formed with pharmaceutically acceptable organic acids such as acetic acid, benzenesulfonic acid, benzoic acid, camphorsulfonic acid, citric acid, ethanesulfonic acid, fumaric acid, glucoheptonic acid, gluconic acid, glutamic 2- acid, glycolic acid, hydroxynaphthoic acid, 2-hydroxyethanesulfonic acid, lactic acid, maleic acid, malic acid, mandelic acid, methanesulfonic acid, muconic acid, 2-naphthalenesulfonic acid, propionic acid, salicylic acid, succinic acid, dibenzoyl-L-tartaric acid, tartaric acid, p-toluenesulfonic acid, trimethylacetic acid, trifluoroacetic acid and the like, or
(3) the pharmaceutically acceptable base addition salts formed when an acid proton present in the parent compound is either replaced with a metal ion, for example an alkali metal ion, an alkaline earth metal ion or an aluminum ion; or coordinated with a pharmaceutically acceptable organic or inorganic base. Acceptable organic bases comprise diethanolamine, ethanolamine, N-methylglucamine, triethanolamine, tromethamine and the like. Acceptable inorganic bases comprise aluminum hydroxide, calcium hydroxide, potassium hydroxide, sodium carbonate and sodium hydroxide.

The expression “enantiomeric mixtures” in the present invention refers to any mixture of enantiomers. The mixture may be racemic, that is to say 50/50 of each enantiomer by weight (w/w), or non-racemic, that is enriched with one or the other of the enantiomers, for example so as to obtain an enantiomeric excess greater than or equal to 95%, preferably greater than or equal to 98%, even more preferably greater than 99%.

The expression “diastereomeric mixtures” in the present invention refers to any mixture of diastereomers regardless of the proportion.

The expression “treatment” applies to all types of animals, preferably to mammals and more preferably to humans. In the case of the treatment of a non-human animal, the expression refers to a veterinary treatment.

The present invention relates to compounds of formula (I):

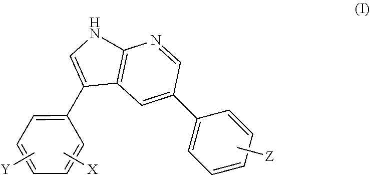

in which
X represents a hydrogen atom or a halogen atom,
Y is selected from a hydrogen atom, a —CH₂OH, —CH═NOH, —CO₂H group, an aryl optionally mono- or polysubstituted, a heteroaryl optionally mono- or polysubstituted, and -L-(CH₂)p-W,
   L represents —C(O)NH—, —CH₂NH—, —NHC(O)—, —CH₂N═CH—, —CH═N—, —NHC(O)NH— or —CH₂NHC(O)—,
   p is an integer from 0 to 2,
   when L represents —CH₂NH—, —NHC(O)—, —CH₂N═CH—, —CH═N—, —NHC(O)NH— or —CH₂NHC(O)—, W is selected from:
   a hydrogen atom,
   a C₁-C₆ alkyl, preferably a methyl,
   a C₆-C₁₀ aryl, optionally mono- or polysubstituted, advantageously by 1 to 3 groups independently selected from a halogen atom, a hydroxyl, a C₁-C₆ alkyl, or a C₁-C₆ alkoxyl, and
   a 5- to 10-membered heteroaryl containing from 1 to 3 heteroatoms independently selected from N, O and S, said heteroaryl optionally being mono- or polysubstituted, advantageously it is optionally substituted with 1 to 4 groups independently selected from:
   a halogen atom,
   a hydroxyl,
   an oxo group
   a C₁-C₆ alkoxyl,
   a C₁-C₆ alkyl, wherein 1 or several hydrogen atoms is optionally replaced with a fluorine atom, and
   a C₆-C₁₀ aryl, optionally substituted with a halogen atom and/or a C₁-C₆ alkyl;
   when L represents —C(O)NH—, W is selected from:
   an optionally mono- or polysubstituted phenyl, advantageously it is optionally substituted with a halogen atom, a hydroxyl group, a C₁-C₆ alkyl, or a C₁-C₆ alkoxyl group, and
   a 5- to 10-membered heteroaryl comprising from 1 to 2 heteroatoms independently selected from N, O and S, said heteroaryl optionally being mono- or polysubstituted, advantageously it is optionally substituted with 1 to 4 substituents independently selected from:
   a halogen atom,
   a hydroxyl,
   an oxo group,
   a C₁-C₆ alkoxyl group,
   a C₁-C₆ alkyl, wherein 1 or several hydrogen atoms is optionally replaced with a fluorine atom, and
   a C₆-C₁₀ aryl, optionally substituted with a halogen atom and/or a C₁-C₆ alkyl;
Z represents —CH₂Q or —O(CH₂)ₘT,
   Q represents —OH, —SO₂R₁, —NR₂R₃, or —R₄
   R₁ represents a C₁-C₆ alkyl,
   R₂ and R₃ each independently represent a C₁-C₆ alkyl, and
   R₄ represents a 5-7 membered heterocycloalkyl, comprising 1 to 2 heteroatoms independently selected from N, O and S, said heterocycloalkyl optionally substituted with 1 to 3 substituents independently selected from a C₁-C₆ alkyl, a C₁-C₆ C(O)-alkyl and a C₁-C₆ C(O)O-alkyl,
   m is equal to 1 or 2,
   T is —O(CH₂)ₙCH₃ or —R₅
   n is 0 or 1
   R₅ is a 5-7 membered heterocycloalkyl, comprising 1 to 2 heteroatoms independently selected from N, O and S, said heterocycloalkyl optionally substituted with 1 to 3 substituents independently selected from a C₁-C₆ alkyl, a C₁-C₆ C(O)-alkyl and a C₁-C₆ C(O)O-alkyl,
a pharmaceutically acceptable salt thereof or a mixture thereof,
for use in the prevention and/or treatment of viral infections.

The compound of formula (I) according to the invention may be in the form of a stereoisomer or a mixture of stereoisomers, such as tautomers, enantiomers or diastereoisomers.

In a first embodiment, the compounds of the invention are of formula (Ia):

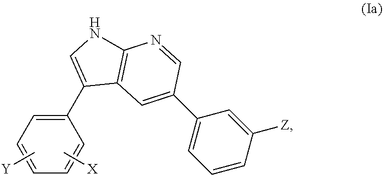

In a second embodiment, the compounds of the invention are of formula (Ib):

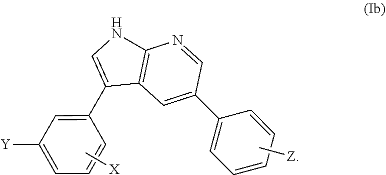

In a third embodiment, the compounds of the invention are of formula (Ic):

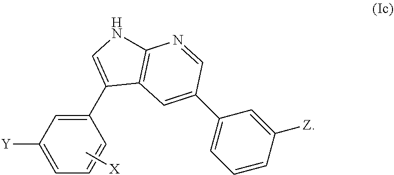

In a fourth embodiment, the compounds of the invention are of formula (Id):

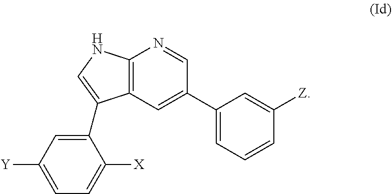

In formulae (Ia), (Ib), (Ic) and (Id), the substituents X, Y and Z are as defined above and below.

In a particular embodiment, in the compounds of the invention, Z represents —CH₂R₄ or —O(CH₂)ₘR₅. Preferably, in this embodiment, R₄ and R₅ independently represent a piperazine, morpholine or homopiperazine group, optionally substituted with a C₁-C₆ alkyl or a C(O)O (C₁-C₆)-alkyl group. In this embodiment, m preferably represents 2.

In a particular embodiment, Z represents —CH₂OH, —CH₂SO₂CH₃, —OCH₂OCH₂CH_{3,} —OCH₂CH₂OCH₃, —N(CH₂CH₃)₂,

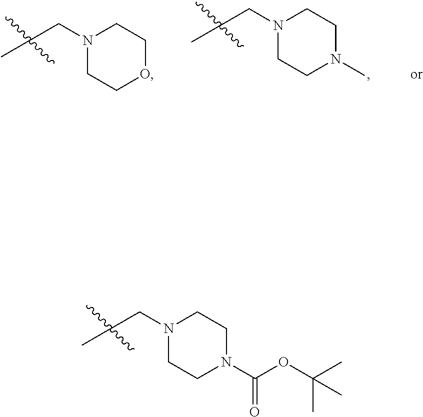

In a preferred embodiment, Z represents —CH₂R₄ wherein R₄ represents a piperazine group, optionally substituted with a C₁-C_{6,} alkyl, in particular a methyl.

In a particular embodiment, X represents a halogen, in particular fluorine.

In a particular embodiment, X represents a hydrogen.

In a particular embodiment, Y represents —H, —CN, —CH₂OH, —CH═NOH, aryl or heteroaryl optionally mono- or polysubstituted, or -L-(CH₂)p-W.

In an advantageous embodiment, Y represents an aryl or heteroaryl, optionally mono- or polysubstituted, or -L-(CH₂)p-W.

In the case where Y represents an aryl or heteroaryl that is optionally mono- or polysubstituted, it is preferably an aryl optionally mono- or polysubstituted, in particular a phenyl, in particular unsubstituted.

In an even more advantageous embodiment, Y represents L-(CH₂)p-W. In the case where Y represents -L-(CH₂)p-W, preferably, p is then equal to 0, 1 or 2.

Advantageously, when Y represents L-(CH₂)p-W, L represents —C(O)NH—, —CH₂NH—, —NHC(O)—, —NHC(O)NH— or —CH₂NHC(O)—, preferably —CH₂NH—, —NHC(O)— or —NHC(O)NH—.

In the embodiment where Y represents -L-(CH₂)p-W, W advantageously represents a C₆-C₁₀ aryl or 5- to 10-membered heteroaryl containing from 1 to 2 heteroatoms independently selected from N, S and O, said aryl or heteroaryl optionally being mono- or polysubstituted.

In a preferred embodiment, W represents a C₆-C₁₀ aryl, in particular a phenyl, unsubstituted or monosubstituted, preferably by a hydroxyl.

In another preferred embodiment, W represents a 5- or 6-membered heteroaryl, containing 1 to 2 heteroatoms independently selected from N, S and O, in particular N, such as pyrazole, pyridine or imidazole. Advantageously, said heteroaryl is optionally substituted with 1 to 4, preferably 1 or 2 groups independently selected from:
a halogen atom, in particular fluorine,
an oxo group,
a C₁-C₆ alkyl, wherein 1 or several hydrogen atoms is optionally replaced with a fluorine atom, in particular a methyl, and
a C₆-C₁₀ aryl optionally substituted with a halogen atom (in particular a fluorine) and/or an alkyl C₁-C_{6,} for example a methyl, a fluorophenyl (preferably 4-fluorophenyl) or a methylfluorophenyl (for example 4-fluoro-2-methylphenyl).

Preferably, W represents pyrazole, 3-oxo-2,3-dihydro-1H-pyrazole, pyridine, 2(1H)-pyridinone, 4(1H)-pyridinone or imidazole, in particular 2(1H)-pyridinone or imidazole, optionally substituted with 1 to 4, preferably 1 or 2 groups independently selected from:
a C₁-C₆ alkyl, in particular a methyl, and
a C₆-C₁₀ aryl optionally substituted with C₁-C₆ alkyl, for example, a methyl.

In particular, W is then chosen from:

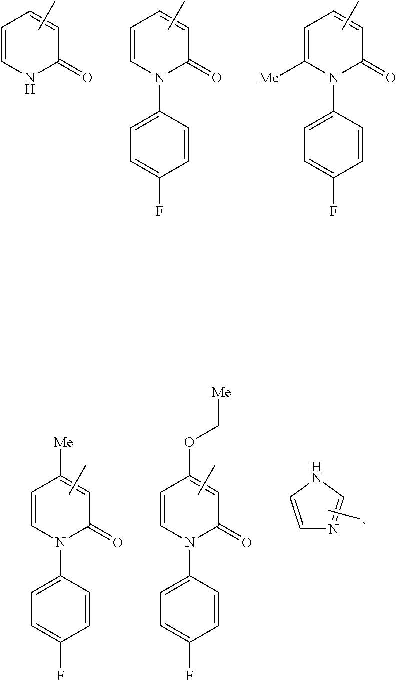

preferably

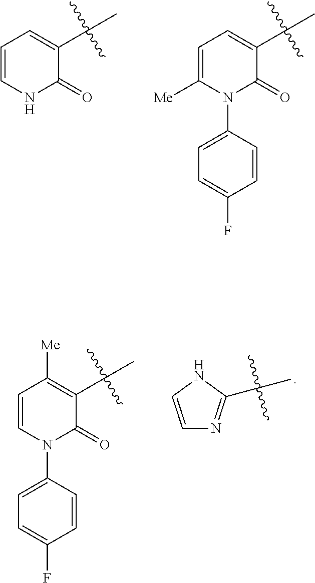 [SMILES: *c1ccc[nH]c1=O ; *c1ccc(C)n(-c2ccc(F)cc2)c1=O ; *c1c(C)ccn(-c2ccc(F)cc2)c1=O ; *c1ncc[nH]1]

In an advantageous embodiment, Y represents an aryl, heteroaryl or L-(CH₂)ₚ—W, L representing —CH₂NH or —NHC(O)NH—, in particular —CH₂NH, p being an integer from 0 to 2, and W being selected from:
a phenyl group optionally substituted with a hydroxyl group, or a C₁-C₆ alkyl group, preferably a hydroxyl, and
a 5- to 10-membered heteroaryl group comprising from 1 to 2 heteroatoms independently selected from N, O and S, in particular N, preferably selected from the group consisting of: pyrrole, imidazole, thiophene, pyridine, pyrimidine, benzofuran, benzodioxole, indole and benzimidazole,

Preferably, when Y represents L-(CH₂)ₚ-W, L representing —CH₂NH or —NHC(O)NH—, in particular —CH₂NH, p being an integer from 0 to 2, W is selected from:
a phenyl group optionally substituted with a hydroxyl group, or a C₁-C₆ alkyl group, preferably a hydroxyl, and
pyrrole, imidazole, 3-oxo-2,3-dihydro-1H-pyrazole, thiophene, pyridine, 2(1H)-pyridinone, 4(1H)-pyridinone, pyrimidine, pyrimidine-2,4-dione, benzofuran, benzodioxole, indole and benzimidazole, more preferably imidazole.

In this embodiment, Z preferably represents —CH₂R₄ or —O(CH₂)ₘ R₅ and preferably —CH₂R₄. Preferably, in this embodiment, R₄ and R₅ independently represent a piperazine or morpholine group, optionally substituted with a C₁-C₆ alkyl or a C(O)O (C₁-C₆)-alkyl group. More preferably, Z represents —CH₂R₄ wherein R₄ represents a piperazine group substituted with a methyl. Furthermore, in this embodiment, X advantageously represents a halogen, in particular fluorine.

In another advantageous embodiment, Y represents —NHC(O)-W, with W representing a 5- to 10-membered heteroaryl group, comprising from 1 to 2 heteroatoms independently selected from N, O and S, in particular N, said heteroaryl optionally being substituted with 1 to 4 substituents independently selected from:
an oxo group,
a C₁-C₆ alkyl wherein 1 or more hydrogen atoms is optionally replaced with a fluorine atom, in particular a methyl, and
a C₆-C₁₀ aryl optionally substituted with a halogen atom (in particular a fluorine) and/or an alkyl C₁-C_{6,} for example a methyl, a fluorophenyl (preferably 4-fluorophenyl) or a methylfluorophenyl (for example 4-fluoro-2-methylphenyl).

Preferably, in this embodiment, the heteroaryl group is selected from the group consisting of: pyrazole, pyrrole, imidazole, thiophene, pyridine, pyrimidine, benzofuran, benzodioxole, indole and benzimidazole, optionally substituted with 1 to 4 substituents independently selected from:
an oxo group,
a C₁-C₆ alkyl, in particular a methyl, and
a C₆-C₁₀ aryl optionally substituted with C₁-C₆ alkyl, for example, a methyl.

Even more preferably, in this embodiment, W is a pyrazole, pyrrole, imidazole, 3-oxo-2,3-dihydro-1H-pyrazole, thiophene, pyridine, 2(1H)-pyridinone, 4(1H)-pyridinone, pyrimidine, pyrimidine-2,4-dione, benzofuran, benzodioxole, indole or benzimidazole, preferably 2(1H)-pyridinone, optionally substituted with 1 to 4 substituents independently selected from:
a C₁-C₆ alkyl group, in particular a methyl, and
a C₆-C₁₀ aryl group, in particular a phenyl optionally substituted with a halogen atom, in particular a fluorine.

In this embodiment, Z preferably represents —CH₂R₄ or —O(CH₂)ₘ R₅, preferably —CH₂R₄. Preferably, in this embodiment, R₄ and R₅ independently represent a piperazine or morpholine group, optionally substituted with a C₁-C₆ alkyl or a C(O)O (C₁-C₆)-alkyl group. More preferably, Z represents —CH₂R₄ wherein R₄ represents a piperazine group substituted with a methyl.

Preferably, the compound of the invention is selected from:

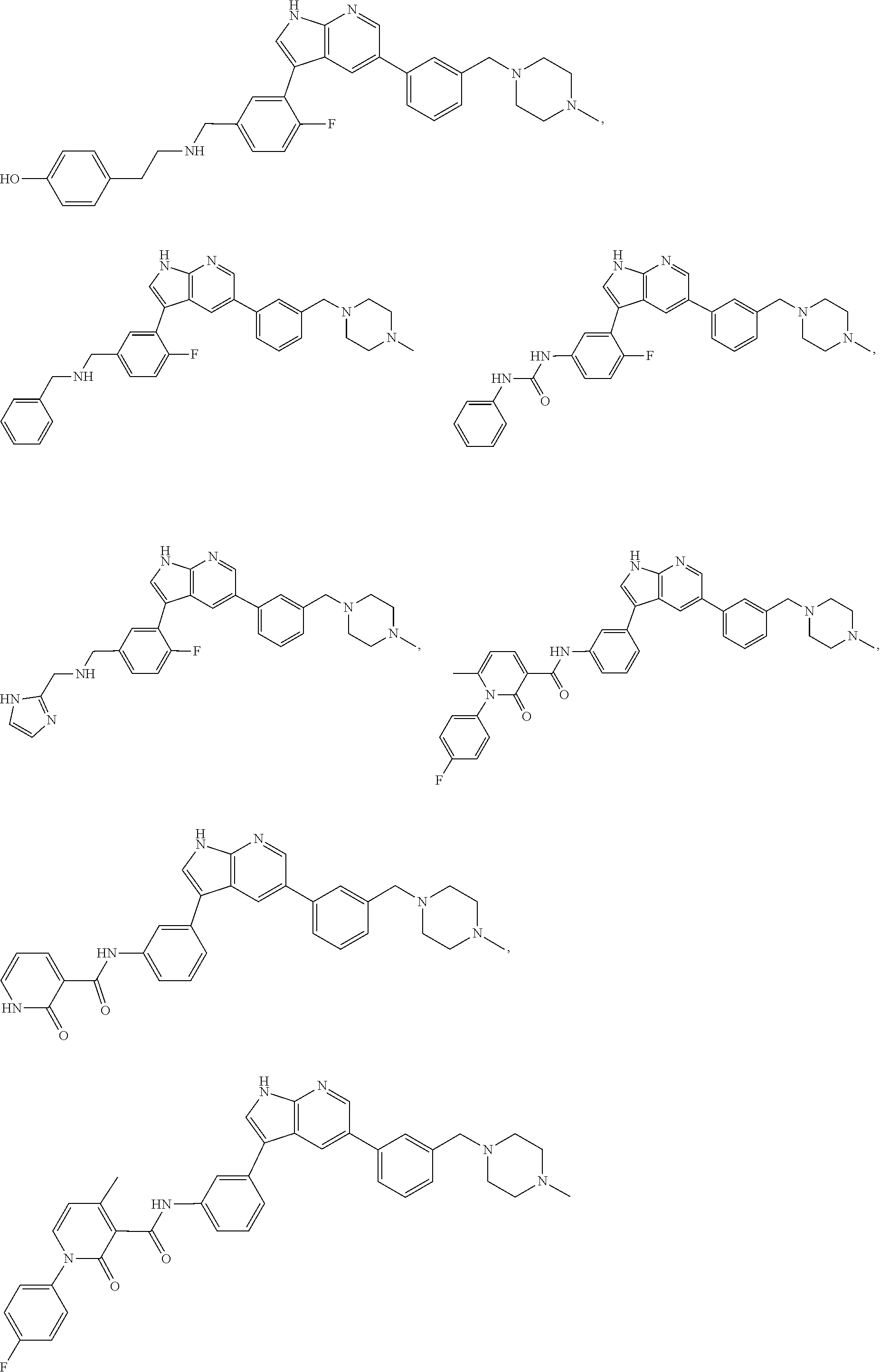

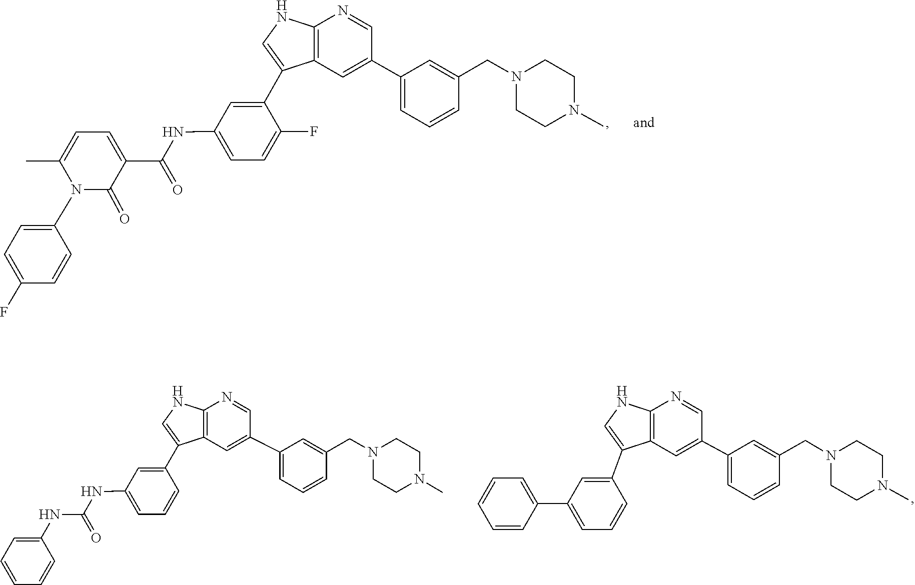

more preferably from among

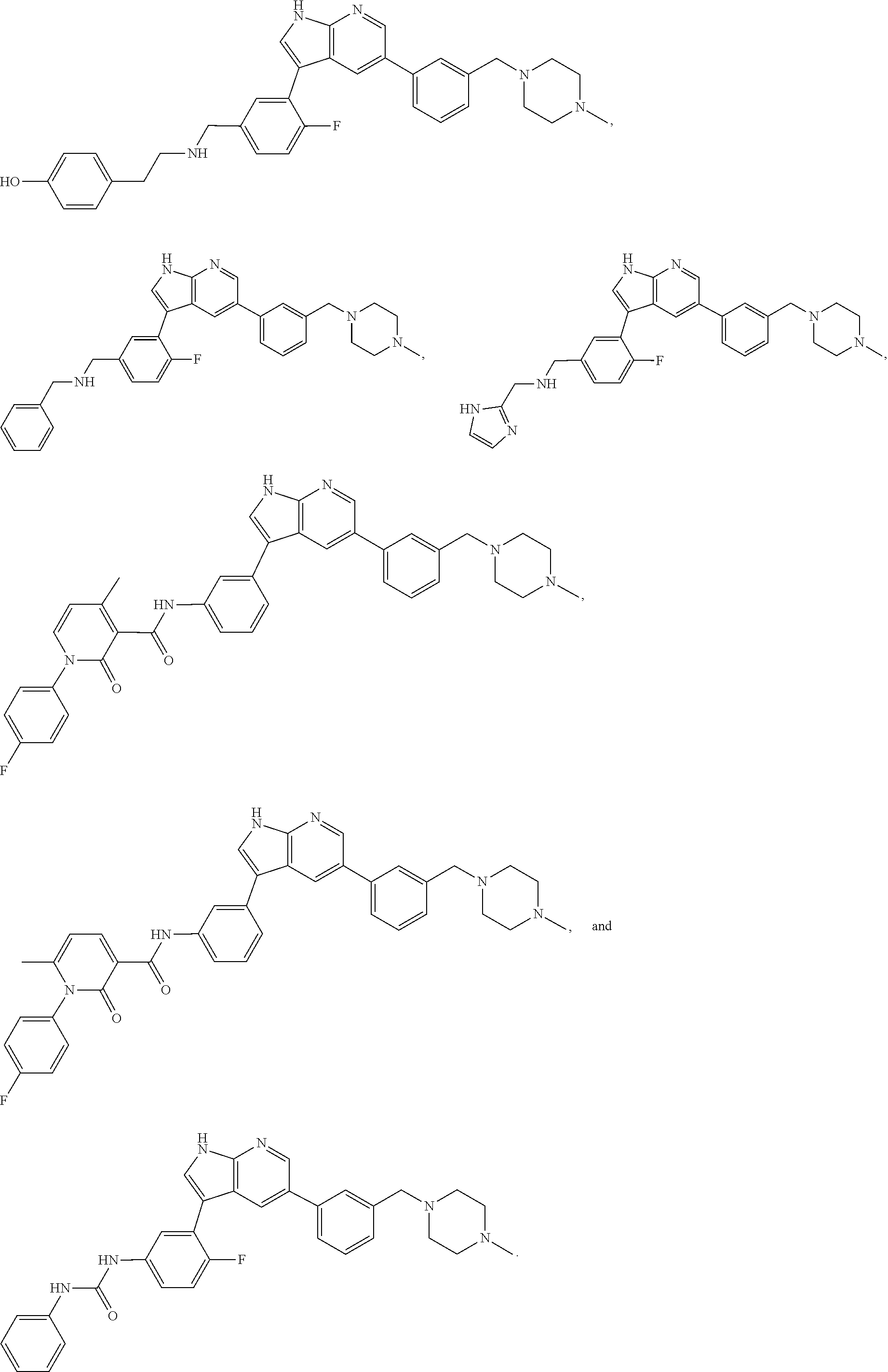 [SMILES: CN1CCN(Cc2cccc(-c3cnc4[nH]cc(-c5cc(CNCCc6ccc(O)cc6)ccc5F)c4c3)c2)CC1 ; CN1CCN(Cc2cccc(-c3cnc4[nH]cc(-c5cc(CNCc6ccccc6)ccc5F)c4c3)c2)CC1 ; CN1CCN(Cc2cccc(-c3cnc4[nH]cc(-c5cc(CNCc6ncc[nH]6)ccc5F)c4c3)c2)CC1 ; Cc1ccn(-c2ccc(F)cc2)c(=O)c1C(=O)Nc1cccc(-c2c[nH]c3ncc(-c4cccc(CN5CCN(C)CC5)c4)cc23)c1 ; Cc1ccc(C(=O)Nc2cccc(-c3c[nH]c4ncc(-c5cccc(CN6CCN(C)CC6)c5)cc34)c2)c(=O)n1-c1ccc(F)cc1 ; CN1CCN(Cc2cccc(-c3cnc4[nH]cc(-c5cc(NC(=O)Nc6ccccc6)ccc5F)c4c3)c2)CC1]

### Compositions and Therapeutic Applications

The compounds of the present invention inhibit AXL receptors. As AXL is involved in numerous biological processes and is a therapeutically validated target, the compounds of the present invention and their pharmaceutically acceptable salts, or the compositions of the invention as defined below, are useful as a medicament, in particular in the treatment of viral infections, the infectious cycle of which depends on AXL and the associated signaling.

The present invention relates to compounds of formula (I) as defined above for use in the prevention and/or treatment of viral infections.

More particularly, the compounds may be used to prepare pharmaceutical compositions comprising, as an active ingredient, at least one compound of formula (I) described above or a pharmaceutically acceptable salt thereof, with at least one pharmaceutically acceptable excipient. Thus, the present invention relates to a pharmaceutical composition comprising a compound of formula (I) according to the invention or a pharmaceutically acceptable salt thereof as an active ingredient, and a pharmaceutically acceptable excipient, for use in the prevention and/or treatment of viral infections. Said excipients are chosen according to the pharmaceutical form and the mode of administration desired from the usual excipients which are known to the person skilled in the art.

The pharmaceutical composition according to the invention may further comprise an additional therapeutic agent, typically chosen from an antiviral agent, an anti-inflammatory agent, an immunomodulatory or immunosuppressive agent, an agent for the treatment of immunodeficiency disorders and an agent for pain treatment. In particular, it is an agent useful in a treatment against viral infections.

One object of the invention therefore relates to the use of the compounds of formula (I) as defined above or of a pharmaceutical composition as defined above in the prevention and/or treatment of viral infections.

In other words, the invention relates to the use of a compound of formula (I) as defined above or of a pharmaceutical composition as defined above for the preparation of a medicament for the prevention and/or treatment of viral infections.

The compounds of formula (I) according to the invention or the pharmaceutically acceptable salts thereof, or the pharmaceutical composition according to the invention are particularly useful for the prevention and/or treatment of viral infections due to Flaviviruses (including dengue virus, Zika virus, West Nile virus, Kunjin virus), Alphaviruses (including Chikungunya virus, Mayaro virus, Semliki Forest virus, Sindbis virus, Eastern equine encephalitis virus, Western equine encephalitis virus, Venezuelan equine encephalitis virus), Filoviruses (including Ebola virus, Marburg fever virus), arenaviruses (including Lassa fever virus, Junin virus, Amapari virus), picornaviruses (including vesicular stomatitis virus), paramyxoviruses (influenza virus), or coronaviruses such as SARS-COV-2.

In a particular embodiment, the invention relates to the compounds of formula (I) as defined above or the pharmaceutical composition as defined above in the prevention and/or treatment of viral infections due to coronavirus, in particular SARS-COV-2.

In this embodiment, the compound of formula (I) preferentially corresponds to the following compounds:

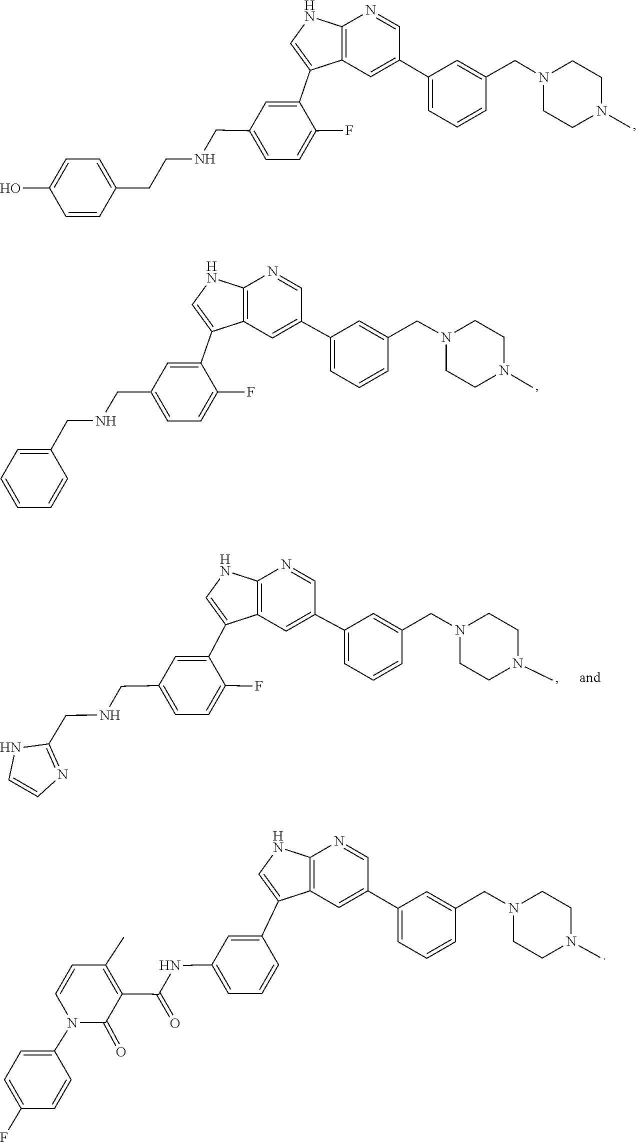 [SMILES: CN1CCN(Cc2cccc(-c3cnc4[nH]cc(-c5cc(CNCCc6ccc(O)cc6)ccc5F)c4c3)c2)CC1 ; CN1CCN(Cc2cccc(-c3cnc4[nH]cc(-c5cc(CNCc6ccccc6)ccc5F)c4c3)c2)CC1 ; CN1CCN(Cc2cccc(-c3cnc4[nH]cc(-c5cc(CNCc6ncc[nH]6)ccc5F)c4c3)c2)CC1 ; Cc1ccn(-c2ccc(F)cc2)c(=O)c1C(=O)Nc1cccc(-c2c[nH]c3ncc(-c4cccc(CN5CCN(C)CC5)c4)cc23)c1]

The present invention also relates to a kit comprising:
a) a first composition comprising a compound according to the invention or a pharmaceutically acceptable salt thereof as an active ingredient, and a pharmaceutically acceptable excipient, and
b) a second composition comprising an additional therapeutic agent, typically selected from an antiviral agent, an anti-inflammatory agent, an immunomodulatory or immunosuppressive agent, an agent for treating immunodeficiency disorders and an agent for pain treatment, preferably an agent useful in treatment against viral infections, as a combination product for separate, concomitant or sequential use.

Said kit is useful as a medicament, in particular for the prevention and/or treatment of viral infections, in particular viral infections as defined above.

The pharmaceutical compositions according to the invention can be administered parenterally, such as by an intravenous or intradermal route, or topically, orally or nasally.

Forms that can be administered parenterally include aqueous suspensions, isotonic saline solutions or sterile and injectable solutions which may contain pharmacologically compatible dispersion and/or wetting agents. Forms that can be administered orally include tablets, soft or hard gel capsules, powders, granules, oral solutions and suspensions. Forms that can be administered nasally include aerosols. Forms that can be administered topically include patches, gels, creams, ointments, lotions, sprays, and eye drops.

Preferably, the compounds or compositions of the invention are administered orally or parenterally (in particular intravenously).

The effective dose of a compound of the invention varies as a function of numerous parameters such as, for example, the chosen administration route, the weight, the age, the sex, the state of progress of the pathology to be treated and the sensitivity of the individual to be treated.

The present invention, according to another of its aspects, also relates to a method for preventing and/or treating the pathologies indicated above which comprises the administration, to a patient in need thereof, of an effective dose of a compound of formula (I) according to the invention, or a pharmaceutically acceptable salt thereof or of a composition according to the invention, preferably parenterally (in particular intravenously) or orally.

### Method for Preparing the Compounds of the Invention

The present invention also relates to methods for preparing the compounds described above, in particular from 5-Bromo-3-iodo-1H-pyrrolo[2,3-b]pyridine.

According to the first embodiment, the method concerning the invention is shown in Diagram 1.

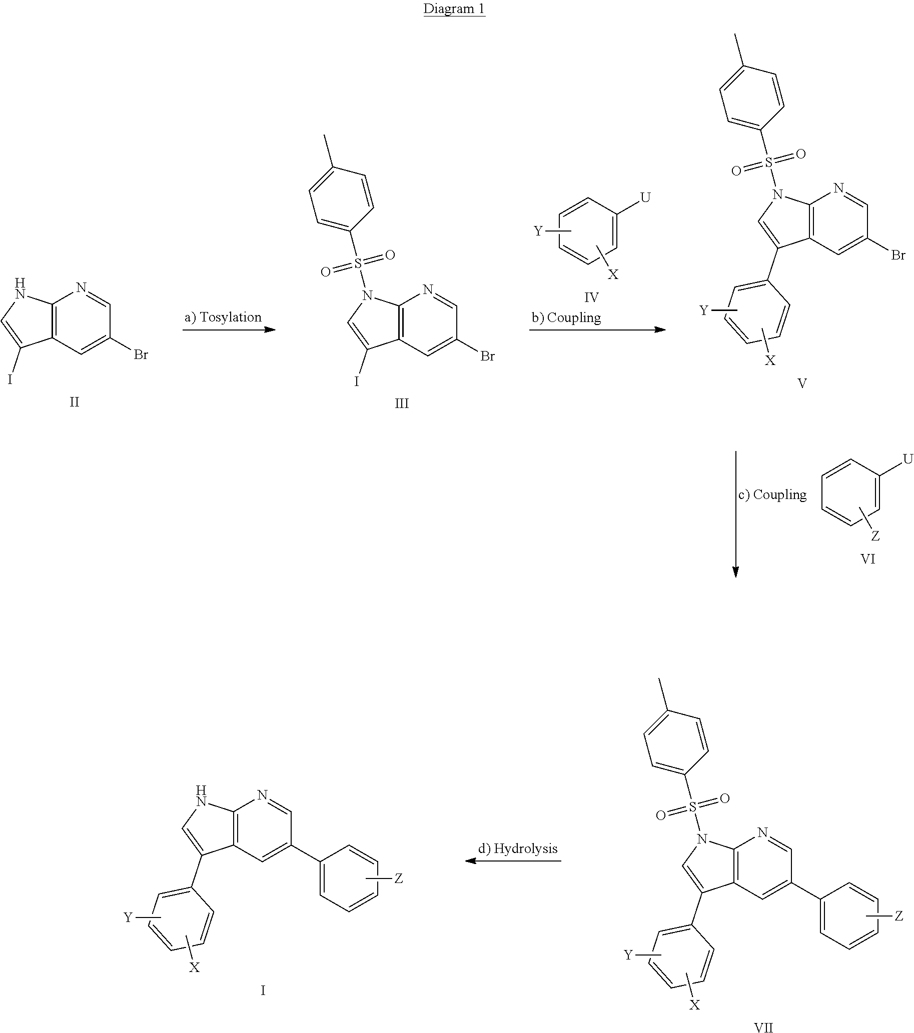

where X and Z are as defined above and Y represents a hydrogen atom, CHO, CN, CH₂OH, CO₂H, NH₂, or a phenyl group.

The method then comprises at least the steps of:
a) tosylation of 5-bromo-3-iodo-1H-pyrrolo[2,3-b]pyridine (II), for example with toluene-4-sulfonyl chloride in the presence of a base such as sodium hydride, to obtain intermediate (III),
b) coupling reaction of the Suzuki-Miyaura type in the presence of a palladium catalyst such as palladium dichloro [1,1′-Bis(diphenylphosphino)ferrocene] (Pd (dppf)Cl₂), with the intermediate of formula (IV) wherein U represents a boronic acid or its pinacol ester, to obtain the compound of formula (V),
c) coupling reaction of the Suzuki-Miyaura type in the presence of a palladium catalyst such as palladium dichloro [1,1′-Bis(diphenylphosphino)ferrocene] (Pd(dppf)Cl₂), with the intermediate of formula (VI) wherein U represents a boronic acid or its pinacol ester, to obtain the intermediate of formula (VII), and
d) hydrolysis of the tosyl group by a base, preferably sodium hydroxide or cesium carbonate, to obtain a compound of formula (I).

The synthesis of the intermediate amine compounds (VIII) (i.e. the compounds of formula (I) for which X and Z are as defined above and Y represents a group (CH₂)ₙNH₂ where n is 1 is represented in Diagram 2.

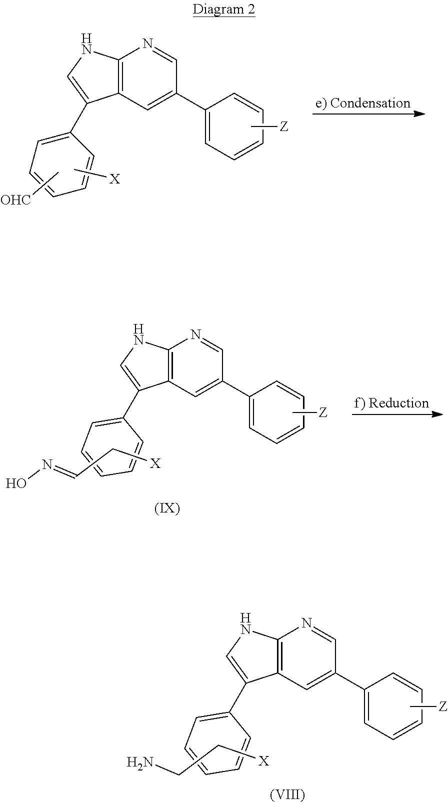

where X and Z are as defined above.

In this embodiment, the method for preparing the amino intermediates of formula (VIII) may comprise at least the steps of:
e) condensing the hydroxylamine on a compound of formula (I) wherein Y is CHO to form the oxime of formula (IX),
f) reducing oxime (IX), in particular in the presence of zinc under ultrasound, to obtain the methylamine intermediate of formula (VIII)

A person skilled in the art will naturally apply all the other synthetic techniques that have been properly described and known to synthesize these types of compounds.

The compounds of formula (I) in which Y represents a L-(CH₂)ₚ-W group with L representing an amide group —NHC(O)— or —CH₂NHC(O)—, are for example obtained by a method of synthesis from the amino-7-azaindole derivatives represented in Diagram 3:

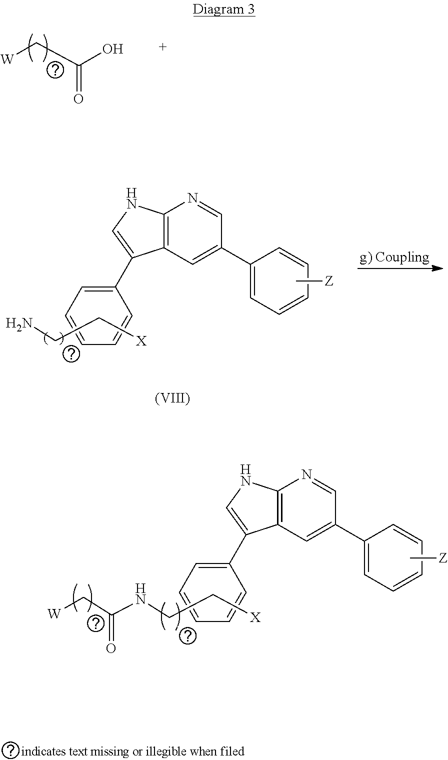

where W, X, Z and p are as defined above and n equal to 0 or 1.

The method of Diagram 3 comprises at least one step of reaction of peptide coupling between an acid of formula W—(CH₂)ₚ—C(O)OH and the amino intermediate of formula (VIII) as defined above, in particular in the presence of at least one activating agent such as 2-(7-aza-1H-benzotriazol-1-yl)-N,N,N′,N′-tetramethyluronium (HATU) and a base such as diisopropylethylamine (DIEA).

A person skilled in the art will naturally apply all the other synthetic techniques that have been properly described and known to obtain these types of amide compounds.

The compounds of formula (I) in which Y represents a L-(CH₂)ₚ-W group with L representing a —CONH— group, are for example obtained by a method of synthesis from carboxylic 7-azaindole derivatives of formula (X) represented in Diagram 4 according to two methods among others:

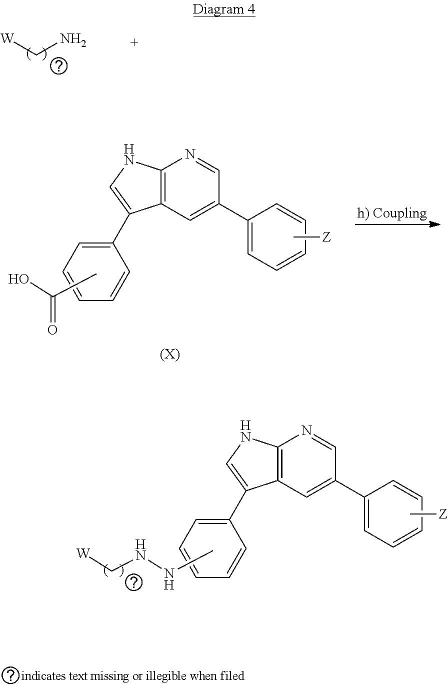

where W, Z, and p are as defined above.

Advantageously, the methods of Diagram 4 comprise at least one step of peptide coupling reaction between an acid of formula (X) and an amine of formula W—(CH₂)ₚ—NH₂, either by coupling or by in situ generating of acyl chlorides by prior action of thionyl chloride.

The urea compounds of the present invention, I.e. the compounds of formula (I) in which Y represents a group L-(CH₂)ₚ-W with L representing a urea group —NHCONH—, are for example prepared according to the method represented in Diagram 5:

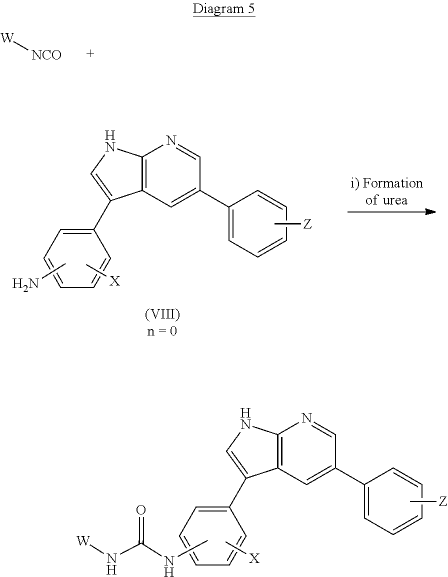

where W, X and Z are as defined above.

Advantageously, the methods of Diagram 5 comprise at least one reaction of a compound of formula (VIII) in which n is equal to 0, with an isocyanate of formula W—NCO, W being as defined above. Said isocyanates are either commercially available, or obtained according to synthesis methods known to a person skilled in the art. Furthermore the person skilled in the art will naturally apply all the other synthetic techniques that have been properly described and known to obtain these types of urea compounds.

According to another embodiment, concerning the method of synthesis of the secondary amine compounds of the present invention, I.e. the compounds of formula (I) in which Y represents a group L-(CH₂)ₚ-W with L representing a group —CH₂NH—, three methods among others are represented in Diagram 6:

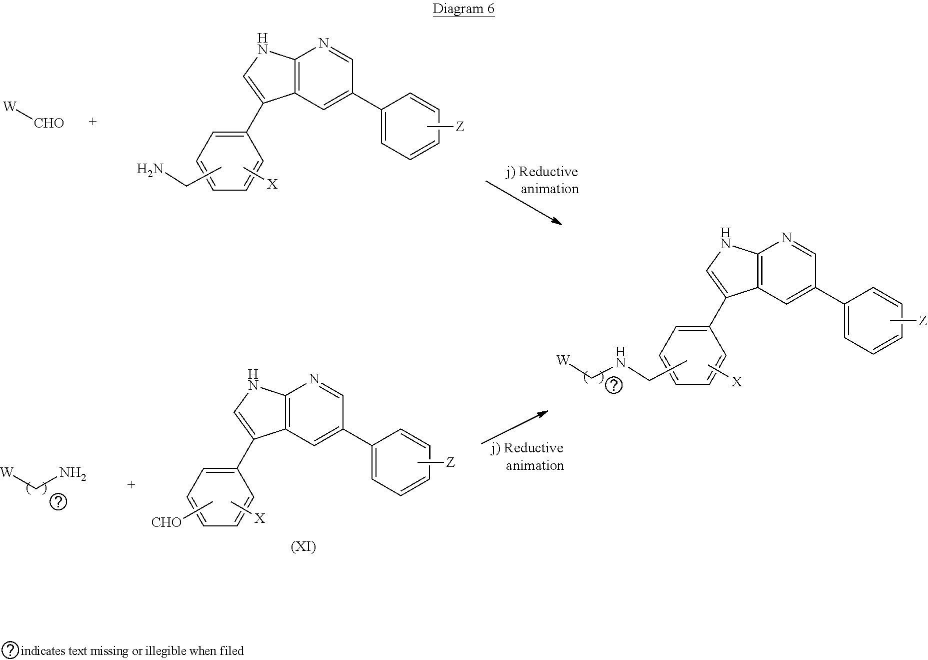

where W, X, Z, and p are as defined above.

Advantageously, the methods of Diagram 6 comprise at least one step of reductive amination between either a compound of formula (VIII) in which n is equal to 1, with aldehydes of formula W—CHO, or between the 7-azalindole aldehydes of formula (XI) and an amine of formula W(CH₂)ₚ—NH₂.

The aldehydes of formula W—CHO and the amines of formula W-(CH₂)ₚ—NH₂ are in particular commercially available, or easily obtained according to preparation methods known by a person skilled in the art.

A person skilled in the art will naturally apply all the other synthetic techniques that have been properly described and known to obtain these types of amide compounds.

Another embodiment relates to the method for synthesizing secondary amine derivatives of formula (XII) obtained by deprotecting the compound of formula (XIII) according to Diagram 7 below:

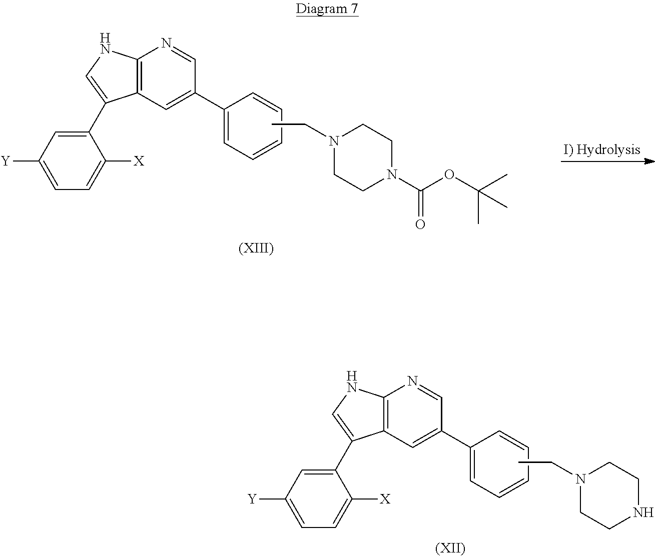

where X and Y are as defined above.

Advantageously, the method comprises at least one step of hydrolysis of the carbamate compound of formula (XIII) in an acid medium to form the desired secondary amine of formula (XII).

A person skilled in the art will naturally apply all other well-known synthetic techniques to deprotect the expected secondary amine.

### DESCRIPTION OF THE FIGURES

FIG. 1: Inhibition of the phosphorylation of Axl in cellulo by compounds 1, 2 and 3 in comparison with R428/Bemcentinib. The A549 cells were preincubated for 1 hour with 2.5 μM of compounds targeting Axl. They were then stimulated for 5 min with 1 mM of pervanadate. The evaluation of the inhibition of the phosphorylation of the Y779 residue of the intracytoplasmic domain of Axl kinase is carried out by immunoblot using the antibody R&D Systems (AF2228) and measuring the intensity of the signals by densitometry (Image J) (P-Axl/Axl ratio).

FIG. 2: Determination of the IC50 of compounds 1, 2 and 3 with respect to the inhibition of the kinase Axl and of the kinase FLT3. 10 concentrations of the compound were prepared by serial dilutions. The compounds are screened in DMSO at 1% final. The kinases were diluted in a concentrated concentration twice in kinase buffer. All the ATP solutions were diluted in a 4-fold concentrated working concentration (50 mM HEPES pH 7.5, 0.01% BRIJ-35, 10 mM MgCl2, 1 mM EGTA). Each compound was incubated at 10 concentrations between 100 nM and 0.00495 nM in order to calculate the IC₅₀. The IC50 are indicated: a) results of compound 1; b) results of compound 2; c) results of compound 3.

FIG. 3: Anti-ZIKV activity of compounds 1, 2, 3, 10 and 11 in comparison with R428/Bemcentinib. The cells were preincubated for 1 hour with the compounds at the indicated concentrations, then infected with the viral strain (MOI=0.1). The cells were kept in culture for 24 hours in the presence of the compound. The residual viral replication is quantified by qRT-PCR of viral genomic RNA. The values are expressed as a percentage of the infection detected in the control condition obtained by incubation of cells in the presence of the dilution solvent DMSO (0). The inhibitor of Axl R428/Bemcentininb is evaluated in parallel. The values are averages of triplicates+standard deviation: a) results for compounds 1, 2, 3, 10 and R428 at a concentration of 2.5 μM; b) results for compounds 1, 2 and R428 at the concentrations of 0.15, 0.31, 0.62, 1.25, 2.5 and 5 μM; c) results for compound 10 at concentrations of 0.1, 0.2, 0.4, 0.5, 1 and 2.5 M and compound 11 at the concentrations of 0.001, 0.1, 0.4, 0.5, and 1 μM d) logarithmic effects of the dose effects obtained for compounds 1 (on the left) and 10 (on the right). The results obtained with R428 are presented in parallel.

FIG. 4: Anti-DENV activity of compounds 10, 11 and 14. The cells were preincubated for 1 hour with the compounds at the indicated concentrations (0.01, 0.1, 0.5, 1 and 2.5 μM), then infected with the viral strain DENV2 (MOI=0.1). The cells were kept in culture for 3 days in the presence of the compound. Residual viral replication, measured by quantification of the intracellular viral RNAs by RT-PCR, the values are expressed as a percentage of the infection detected in the control condition obtained by incubation of cells in the presence of the dilution solvent DMSO (0). The values are averages of triplicates+standard deviation.

FIG. 5: Anti-KUNV activity of compound 10. The cells were preincubated for 1 hour with the compounds at the indicated concentrations (0.001, 0.1, 0.5, 1 and 2.5 μM), then infected with the viral strain (MOI=0.1). The cells were kept in culture for 24 hours in the presence of the compound. Residual viral replication is measured by quantification of luciferase activity in the cell lysate is expressed as a percentage of the infection detected in the control condition obtained by incubation of cells in the presence of the dilution solvent DMSO (0). The values are averages of triplicates+standard deviation.

FIG. 6: Anti-CHIKV activity of compound 10. The cells were preincubated for 1 hour with the compounds at the indicated concentrations (0.001, 0.1, 0.25, 0.5, 1 and 1.5 μM), then infected with the viral strain LR-OPY-1 (MOI=0.1). The cells were kept in culture for 24 hours in the presence of the compound. Residual viral replication, measured by quantification of luciferase activity in the cell lysate, is expressed as a percentage of the infection detected in the control condition obtained by incubation of cells in the presence of DMSO (0). The values are averages of triplicates+standard deviation.

FIG. 7: Anti-SARS-COV-2 activity of compounds 1, 2, 3, 10 and 14 determined in the VeroE6 line. The cells were preincubated for 1 hour with compounds at a concentration of 1.25 μM, then infected with the viral strain BetaCoV/France/IDF0371/2020 (MOI=0.01). The cells were kept in culture for 24 hours in the presence of the compound. The residual viral replication is measured by quantification of the viral RNA in the cell culture by qRT-PCR. The control condition is obtained by incubation of cells in the presence of the dilution solvent DMSO (0). The values are averages of triplicates+standard deviation.

FIG. 8: Anti-SARS-COV-2 activity of compounds 1, 2, 3, 10 and 14 determined in A549-ACE2 cells. The cells were preincubated for 1 hour with compounds at a concentration of 1.25 UM, then infected with the viral strain BetaCoV/France/IDF0371/2020 (MOI=0.01). The cells were kept in culture for 24 hours in the presence of the compound. The residual viral replication is measured by quantification of the viral RNA in the cell culture by qRT-PCR. The control condition is obtained by incubation of cells in the presence of the dilution solvent DMSO (0). The inhibitor of Axl R428/Bemcentininb is evaluated in parallel. The values are averages of triplicates+standard deviation.

FIG. 9: Calculation of the specific activities of compounds 1, 2 and 3 on SARS-COV-2. The A549-ACE2 cells were incubated with increasing concentrations of compounds for 1 hour, then infected with the viral strain BetaCoV/France/IDF0371/2020 (MOI=0.01). The cells were kept in culture for 24 hours in the presence of the compound. The residual viral replication is measured by quantification of the viral RNA in the cell culture by qRT-PCR. The control condition is obtained by incubation of cells in the presence of DMSO. The values are averages of triplicates+standard deviation. The IC50 are calculated using the software Prism (GraphPad).

FIG. 10: Anti-SARS-COV-2 activity of compound 1 determined in a primary human bronchial epithelium model. The cells were preincubated for 1 hour with the compounds used at the concentration of 1.25 μM, added to the basal compartment of the culture (MucilAir™, Epielix). The cells were then infected by the viral strain BetaCoV/France/IDF 0371/2020 added to the apical compartment (MOI=0.1). After 2 hours, the inoculum is removed and the cells are kept in culture for 24 hours in the presence of the compound. The residual viral replication is measured by quantification of the viral RNA in the cell culture by qRT-PCR. The control condition is obtained by incubation of cells in the presence of the dilution solvent DMSO (0). NI indicates the condition corresponding to uninfected cells. The individual values as well as averages and standard deviations are indicated.

FIG. 11: Mechanism for inhibiting A549-ACE2 cell infection by compound 1. The A549-ACE2 (a) or veroE6 (b) cells were infected by SARS-COV-2 (BetaCoV/France/IDF0371/2020 strain) (MOI=0.01). Compound 1 (1.25 μM) was added to the cell culture either 1 hour before the viral challenge and maintained throughout the experiment (Diagram 1, condition SARS-COV-2); 1 hour before the viral challenge and only during the first 4 hours of the infection (Diagram 2, condition Pre-Tt); from the 4th hour following exposure to SARS-COV-2 (Diagram 3, condition Post-Tt). The infection of the cells is determined by intracellular detection of the Spike protein by FACS using specific antibodies (anti-Spike SARS-COV-2; clone 1A9, Gene Tex). The individual values as well as the averages and the standard deviations are indicated and compared with the control condition carried out from cells maintained in the presence of dilution solvent DMSO (0).

### EXAMPLES

The invention will be better understood upon reading the following examples, which are given purely by way of illustration and should not be interpreted as limiting the scope of the present invention.

### Example 1: Synthesis

### Material

The syntheses and analyses were carried out under the following conditions. Magnetic Nuclear Resonance ¹H and ¹³C:

Apparatus: Bruker Avance 400 (400 MHZ); Bruker Avance 300 (300 MHZ)

Conditions of use: Ambient temperature, chemical shifts expressed in parts per million (ppm), multiplicity of signals indicated by lowercase letters (singlet s, doublet d, triplet t, quadruplet q, multiplet m), dimethyl sulphoxide de, methanol d4, chloroform d1 as deuterated solvents.

High-Pressure Liquid Chromatography (HPLC):

Apparatus: Agilent Technology 1260 Infinity

Conditions of use: Zorbax SB-C18 column or Eclipse plus (2.1×50 mm), 1.8 μm; temperature: 30° C., flow rate: 0.5 mL/min for the methods X and Y and 0.4 mL/min for the method Z, elution gradient Water (A) /Acetonitrile (B) /Formic acid 0.1% (Time (min)/% B):

Method X: 0/10, 0.3/10, 5.7/100, 6.0/100

Method Y: 0/10, 1/50, 6/100, 8/100

Method Z: 0/10, 11/100, 15/100

Mass Spectrometry (MS):

Apparatus: Quadripole Agilent Technologies 6120

Conditions of use: ElectroSpray (ESI) in positive and/or negative mode.

Weighing:

Appliance: Denver Instrument TP214 (0.1 mg precision)

Conditions of use: Weighing carried out to the nearest milligram.

Pressure reactions:

Apparatus: Autoclave Parr 300 mL.

Conditions of use: Hydrogenation under 20 bars of hydrogen.

Reaction under microwave irradiation:

Appliance: CEM Discover SP®

Conditions of use: Power of 200 Watts, Average stirring speed

In the following, the following abbreviations are used:

| | | | |
|---|---|---|---|
| eq | equivalent | DMF | N,N-dimethylformamide |
| RT | Room Temperature | DMSO | dimethylsulfoxide |

### General Synthesis of 3,5-diaryl-1H-pyrrolo[2,3-b]pyridines (I) Intermediates

### 1ST step, synthesis of 5-bromo-3-iodo-1-(toluene-4-sulfonyl)-1H-pyrrolo[2,3-b]pyridine (III)

3G of 5-bromo-3-iodo-1H-pyrrolo [2,3-b] pyridine (II) are diluted in 60 ml of anhydrous THF under argon and cooled in an ice bath. 558 mg (1.5 eq) of sodium hydride (60%) are added slowly and the medium is stirred at 0° C. for 20 minutes. Then tosyl chloride (2.11 g, 1.2 eq) is added, and the medium is stirred at RT for 5 hours. The solvent is evaporated. The residue obtained is suspended in a minimum of petroleum ethers and filtered. The precipitate is washed twice with 2M sodium hydroxide solution, then dried under vacuum overnight.

RMN ¹H (400 MHZ, DMSO-d6) δ (ppm) 8.52 (d, J=1.9, 1H), 8.22 (s, 1H), 8.01 (m, 3H), 7.44 (d, J=8.2, 2H), 2.51 (s, 3H).

Yield: 97%; HPLC: 99%; MS [M+1]: 476.9-478.9

### 2nd Step, General Synthesis of 3-5-aryl-1H-pyrrolo[2.3-b]pyridines (I) (in Which Y=CHO, NH₂ or a Phenyl Substituted by a Phenyl)

5-bromo-3-iodo-1-(toluene-4-sulfonyl)-1H-pyrrolo [2,3-b] pyridine (III) (100 mg), the first boronic acid to be coupled (IV) (1 eq) and potassium carbonate (3 eq) are suspended in a 3/1 dioxane/water mixture (3 mL), and the mixture is degassed under argon for 20 minutes. Pd(dppf)Cl₂ (2%) is added and the mixture is stirred at 80° ° C. under microwave irradiation for 1 to 3 times 20 minutes until the starting reagent is completely consumed. The reaction medium is washed with an aqueous solution saturated with NaHCO3 and extracted with ethyl acetate. The solvents are evaporated off, the residue is taken up in ethyl acetate, washed with an aqueous solution saturated with NaHCO3, dried over Na2SO4, filtered and evaporated to dryness, and then purified on a silica column. The compound is then dissolved in a dioxane/water (3/1, 4 mL) mixture, in the presence of the second boronic acid or its ester of pinacol (VI) (1.2 eq) and potassium carbonate (3 eq). The medium is degassed again under argon for 20 minutes, Pd(dppf)Cl₂ (0.025 eq) is then added and the mixture is stirred at 120° C. for 45 minutes under microwave irradiation. 500 μL of 1M sodium hydroxide (3 eq) are then added to the reaction medium and stirred at 100° C. for 45 minutes under microwave irradiation. The reaction medium is then washed with an aqueous solution saturated with NaHCO3, extracted with ethyl acetate, dried over anhydrous Na2SO4, filtered and evaporated to dryness, and then purified on a normal phase silica column (dichloromethane/methanol-ammonia).

| | |
|---|---|
| 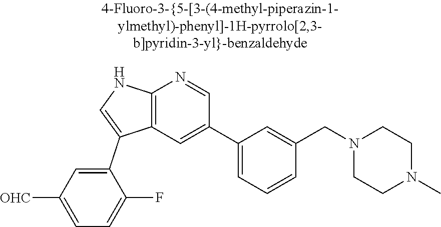 [SMILES: CN1CCN(Cc2cccc(-c3cnc4[nH]cc(-c5cc(C=O)ccc5F)c4c3)c2)CC1] | Reagents: First 2-Fluoro-5- formylbenzeneboronic acid then 1- Methyl-4-[3-(4,4,5,5-tetramethyl-[1,3,2] dioxaborolan-2-yl)-benzyl]-piperazine Yield: 95%; HPLC: 97%; MS [M + 1]: 429.2 |
| 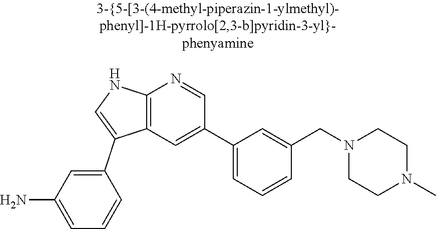 [SMILES: CN1CCN(Cc2cccc(-c3cnc4[nH]cc(-c5cccc(N)c5)c4c3)c2)CC1] | Reagents: First 3-Aminophenylboronic acid then 1-Methyl-4-[3-(4,4,5,5- tetramethyl-[1,3,2]dioxaborolan-2-yl)- benzyl]-piperazine Yield: 66%; HPLC: 98%; MS [M + 1]: 398.3 |
| 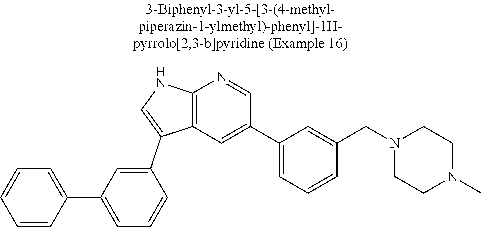 [SMILES: CN1CCN(Cc2cccc(-c3cnc4[nH]cc(-c5cccc(-c6ccccc6)c5)c4c3)c2)CC1] | Reagents: First 3-Biphenylboronic acid then 1-Methyl-4-[3-(4,4,5,5- tetramethyl-[1,3,2]dioxaborolan-2-yl)- benzyl]-piperazine ¹H NMR (400 MHz, DMSO-d6) δ (ppm) 12.06 (s, 1H), 8.56 (d, J = 2.0 Hz, 1H), 8.43 (d, J = 2.0 Hz, 1H), 8.06 (s, 1H), 7.98 (s, 1H), 7.83-7.79 (m, 1H), 7.79-7.75 (m, 2H), 7.67- 7.62 (m, 2H), 7.56 (d, J = 4.9 Hz, 2H), 7.52- 7.36 (m, 4H), 7.30 (d, J = 7.5 Hz, 1H), 3.55 (s, 2H), 2.48-2.20 (m, 7H), 2.13 (s, 3H). Yield: 37%; HPLC: 99%; MS [M + 1]: 459.3 |

### Synthesis of the Amines

### Method 1: Synthesis of 4-Fluoro-3-{5-[3-(4-methyl-piperazin-1-ylmethyl)-phenyl]-1H-pyrrolo[2.3-b]pyridin-3-yl}-phenylamine

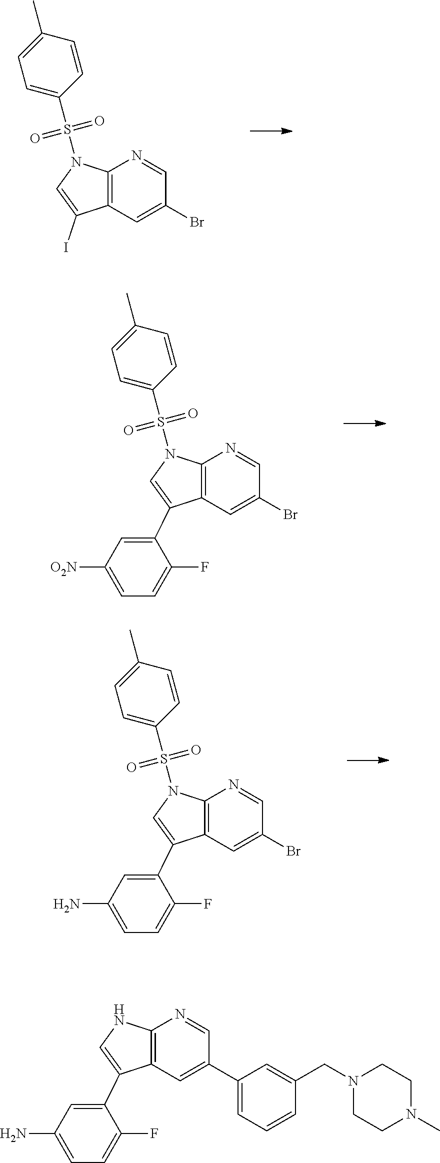

### Step 1: Synthesis of 5-Bromo-3-(2-fluoro-5-nitro-phenyl)-1-(toluene-4-sulfonyl)-1H-pyrrolo[2,3-b]pyridine

5-bromo-3-iodo-1-(toluene-4-sulfonyl)-pyrrolo [2,3-b] pyridine (460 mg), le 2-(2-Fluoro-5-nitro-phenyl)-4,4,5,5-tetramethyl-[1,3,2]dioxaborolane (1 eq) and potassium carbonate (3 eq) are suspended in a dioxane/water mixture (9/1, 20 mL), and degassed under argon for 20 minutes. Pd(dppf)Cl₂ (18 mg, 0.025 eq) is added and the mixture is stirred for 2 to 4 times 20 minutes at 50° C. under microwave irradiation until total consumption of the reagent measured by LC/MS. The solvents are evaporated off, the residue is taken up in ethyl acetate, washed with an aqueous solution saturated with NaHCO3, dried over Na2SO4, filtered and evaporated to dryness, and then purified on a normal-phase silica column (petrol ether/dicholoromethane).

Yield: 66%; HPLC: 90%; MS [M+1]: 490.0-492.0

### Step 2: Synthesis of 3-[5-Bromo-1-(toluene-4-sulfonyl)-1H-pyrrolo[2,3-b]pyridin-3-yl]-4-fluoro-phenylamine

5-Bromo-3-(2-fluoro-5-nitro-phenyl)-1-(toluene-4-sulfonyl)-1H-pyrrolo[2,3-b]pyridine (100 mg) is suspended in 3 mL of ethyl acetate with 232 mg of tin chloride dihydrate (5 eq) and stirred under reflux for 4 hours. The reaction medium is taken up in ethyl acetate and washed with saturated aqueous NaHCO3 solution. The precipitate is filtered off and rinsed with water, then with ethyl acetate. The aqueous phase is extracted with ethyl acetate. The organic phases are dried over Na2SO4, filtered and evaporated to dryness, then purified on a normal-phase silica column (petroleum ether/ethyl acetate).

Yield: 76%; HPLC: 97%; MS [M+1]: 460.0-462.0

### Step 3: Synthesis of 4-Fluoro-3-{5-[3-(4-methyl-piperazin-1-ylmethyl)-phenyl]-1H-pyrrolo[2,3-b]pyridin-3-yl}-phenylamine

3-[5-Bromo-1-(toluene-4-sulfonyl)-1H-pyrrolo[2,3-b]pyridin-3-yl]-4-fluorophenylamine (475 mg), 1-methyl-4-[3-(4,4,5,5-tetramethyl-[1,3,2]dioxaborolan-2-yl)-benzyl]-piperazine (1.2 eq) and potassium carbonate (3 eq) are suspended in a dioxane/water mixture (9/1, 20 mL) and degassed under argon for 20 minutes. Pd(dppf)Cl₂ (19 mg, 0.025 eq) is then added and the mixture is stirred at 120° C. for 45 minutes under microwave irradiation. 3 μL of 1M sodium hydroxide (3 eq) is then added to the reaction medium and stirred at 100° C. for 45 minutes under microwave irradiation. The reaction medium is then washed with an aqueous solution saturated with NaHCO3, extracted with ethyl acetate, dried over anhydrous Na2SO4, filtered and evaporated to dryness, and then purified on a normal phase silica column (dichloromethane/methanol-ammonia.

Yield: 91%; HPLC: 98%; MS [M+1]: 416.3

Synthesis of the various non-commercial carboxylic acids:

1-(4-fluorophenyl)-2-oxo-1,2-dihydropyridine-3-carboxylic acid (HPLC: 97%; MS [M+1]: 243.1) was obtained in accordance with the procedure described [Traore et al. European Journal of Medicinal Chemistry (2013), 70, 789-801].

1-(4-fluorophenyl)-6-methyl-2-oxo-1,2-dihydropyridine-3-carboxylic acid (HPLC: 97%; MS [M+1]: 248.2) was obtained in accordance with the procedure described [Flynn et al. Organic Process Research & Development (2014), 18(4), 501-510]. 1-(4-fluorophenyl)-4-methyl-2-oxo-1,2-dihydro-pyridine-3-carboxylic acid (HPLC: 100%; MS [M+1]: 248.2) was obtained as a by-product during the synthesis of 1-(4-fluorophenyl)-6-methyl-2-oxo-1,2-dihydro-pyridine-3-carboxylic acid described above.

General synthesis of amide compounds from amino-7-azaindole and carboxylic acids compounds.

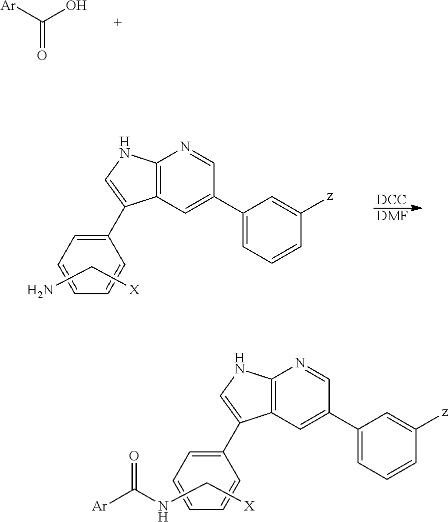

Carboxylic acid (1 eq) is dissolved in anhydrous DMF and placed in dried schlenck, under an Argon atmosphere. N,N′-dicyclohexylcarbodiimide (1 eq), and then the derivative amino-7-azaindole (1 eq) are added. The medium is then stirred at 70° C. overnight. The solvent is evaporated. The residue obtained is then dissolved in ethyl acetate, washed twice with an aqueous solution saturated with NaHCO3, once with an aqueous solution saturated with sodium chloride dried over Na2SO4, filtered on cotton, evaporated to dryness and purified on a reverse-phase silica column (water/acetonitrile) with 1% of trifluoroacetic acid to give the desired amide derivative.

| | |
|---|---|
| 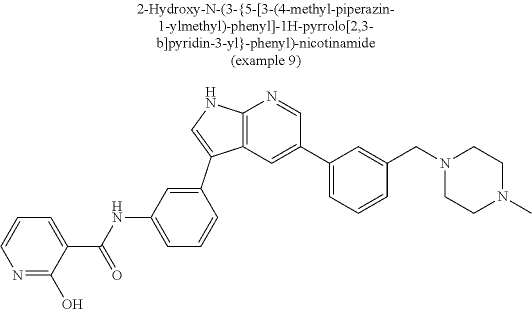 [SMILES: CN1CCN(Cc2cccc(-c3cnc4[nH]cc(-c5cccc(NC(=O)c6cccnc6O)c5)c4c3)c2)CC1] | Reagents: 3-{5-[3-(4-Methyl- piperazin-1-ylmethyl)-phenyl]-1H- pyrrolo[2,3-b]pyridin-3-yl}- phenylamine and 2-Hydroxy- nicotinic acid ¹H NMR (400 MHz, DMSO-d6) δ (ppm) 13.00-12.55 (m, 1H), 12.27 (s, 1H), 12.04 (d, J = 2.2, 1H), 8.59 (d, J = 2.1, 1H), 8.54-8.48 (m, 2H), 8.36-8.30 (m, 1H), 7.95 (d, J = 2.6, 1H), 7.83 (dd, J = 2.2, 6.2, 1H), 7.72- 7.63 (m, 2H), 7.57-7.41 (m, 4H), 7.35-7.28 (m, 1H), 6.59 (dd, J = 6.3, 7.2, 1H), 3.57 (s, 2H), 2.49-2.20 (m, 8H), 2.15 (s, 3H). Yield: 48%; HPLC: 99%; MS [M + 1]: 519.3 |
| 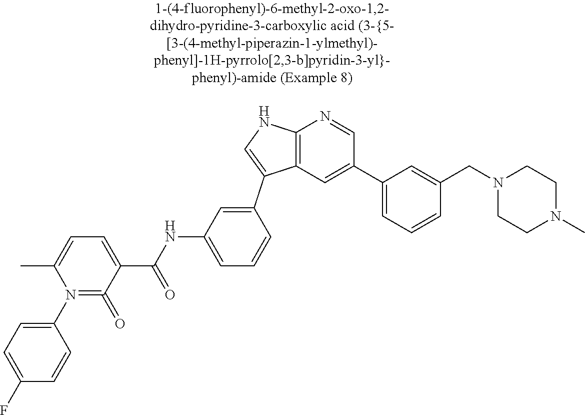 | Reagents: 3-{5-[3-(4-Methyl- piperazin-1-ylmethyl)-phenyl]-1H- pyrrolo[2,3-b]pyridin-3-yl)- phenylamine and 1-(4-Fluoro- phenyl)-6-methyl-2-oxo-1,2- dihydropyridine-3-carboxylic acid ¹H NMR (600 MHz, D₂O) δ (ppm) 8.63 (m, 1H), 8.44 (s, 1H), 7.89 (d, J = 7.0, 1H), 7.71-7.58 (m, 3H), 7.44 (d, J = 7.4, 1H), 7.22-7.08 (m, 6H), 7.08-7.01 (m, 2H), 7.00-6.92 (m, 1H), 6.35 (d, J = 7.5, 1H), 4.22 (s, 2H), 3.89-3.31 (m, 8H), 2.92 (s, 3H), 1.94 (s, 3H). Yield: 67%; HPLC: 99%; MS [M + 1]: 627.3 |
| 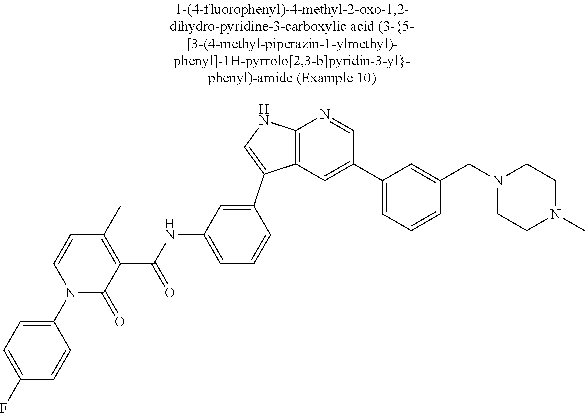 [SMILES: Cc1ccn(-c2ccc(F)cc2)c(=O)c1C(=O)Nc1cccc(-c2c[nH]c3ncc(-c4cccc(CN5CCN(C)CC5)c4)cc23)c1] | Reagents: 3-{5-[3-(4-Methyl- piperazin-1-ylmethyl)-phenyl]-1H- pyrrolo[2,3-b]pyridin-3-yl)- phenylamine and 1-(4-Fluoro- phenyl)-4-methyl-2-oxo-1,2- dihydropyridine-3-carboxylic acid NMR ¹H (400 MHz, DMSO-d6) δ (ppm) 12.02 (d, J = 2.5, 1H), 10.63 (s, 1H), 8.56 (d, J = 1.9, 1H), 8.50 (d, J = 1.9, 1H), 8.24-8.19 (m, 1H), 7.90 (d, J = 2.5, 1H), 7.71 (d, J = 7.0, 1H), 7.66-7.61 (m, 2H), 7.61-7.55 (m, 1H), 7.52-7.34 (m, 7H), 7.30 (d, J = 7.6, 1H), 6.36 (d, J = 7.0, 1H), 3.53 (s, 2H), 2.47-2.16 (m, 11H), 2.12 (s, 3H). Yield: 54%; HPLC: 97%; MS [M + 1]: 627.3 |
| 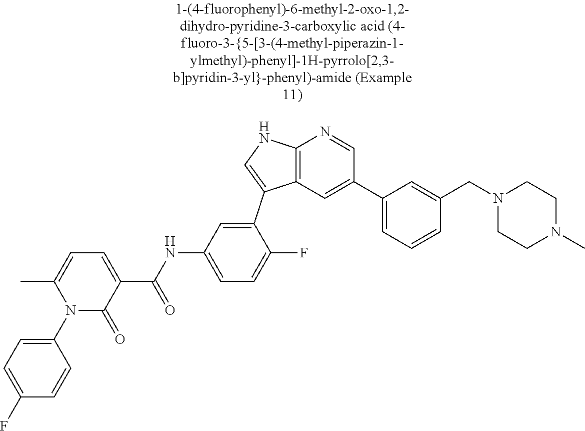 [SMILES: Cc1ccc(C(=O)Nc2ccc(F)c(-c3c[nH]c4ncc(-c5cccc(CN6CCN(C)CC6)c5)cc34)c2)c(=O)n1-c1ccc(F)cc1] | Reagents: 4-Fluoro-3-{5-[3-(4- Methyl-piperazin-1-ylmethyl)- phenyl]-1H-pyrrolo[2,3-b]pyridin-3- yl)-phenylamine and 1-(4-Fluoro- phenyl)-6-methyl-2-oxo-1,2- dihydropyridine-3-carboxylic acid ¹H NMR (400 MHz, DMSO-d6) δ (ppm) 12.16 (d, J = 2.2, 1H), 11.95 (s, 1H), 8.60 (d, J = 2.0, 1H), 8.52 (d, J = 7.5, 1H), 8.40-8.37 (m, 1H), 8.35 (dd, J = 2.6, 7.0, 1H), 7.89- 7.85 (m, 1H), 7.70-7.61 (m, 2H), 7.54-7.40 (m, 6H), 7.35-7.26 (m, 2H), 6.70 (d, J = 7.9, 1H), 3.56 (s, 2H), 2.47-2.21 (m, 8H), 2.13 (s, 3H), 2.07 (s, 3H). Yield: 63%; HPLC: 99%; MS [M + 1]: 645.3 |

### General Synthesis of Ureas

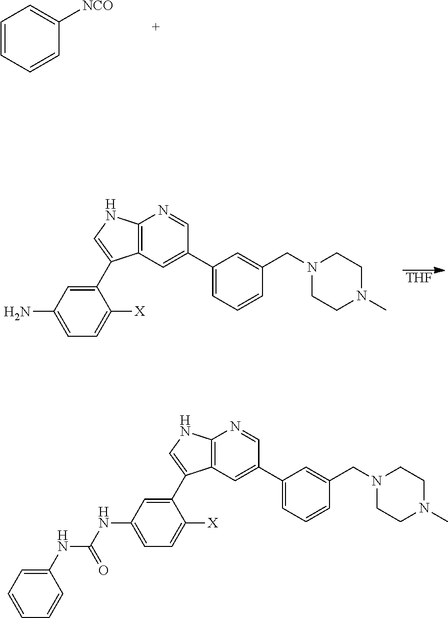

The amino derivative (1 eq), the phenyl isocyanate (1.05 eq) and triethylamine (2 eq) are dissolved in anhydrous THF and stirred, under argon, at room temperature overnight. The reaction medium is quenched with saturated NaHCO3 solution. The aqueous phase is extracted with ethyl acetate and dried over sodium sulfate. The organic phases are combined, evaporated to dryness and purified on a normal-phase silica column (dichloromethane/methanol-ammonia) to give the desired urea derivative.

| | |
|---|---|
| 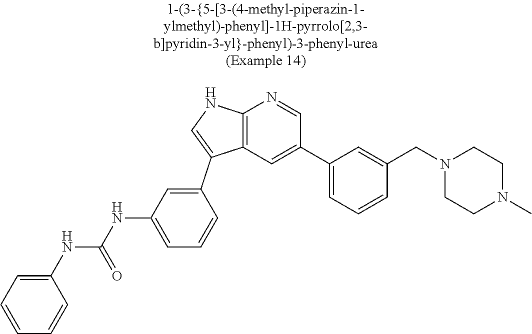 [SMILES: CN1CCN(Cc2cccc(-c3cnc4[nH]cc(-c5cccc(NC(=O)Nc6ccccc6)c5)c4c3)c2)CC1] | Reagents: 3-{5-[3-(4-Methyl- piperazin-1-ylmethyl)-phenyl]-1H- pyrrolo[2,3-b]pyridin-3-yl}- phenylamine and phenyl isocyanate ¹H NMR (400 MHz, DMSO-d6) δ (ppm) 12.01 (d, J = 2.0, 1H), 8.76 (bs, 1H), 8.70 (bs, 1H), 8.57 (d, J = 2.0, 1H), 8.52 (d, J = 1.9, 1H), 8.04- 8.00 (m, 1H), 7.89 (d, J = 2.6, 1H), 7.72-7.62 (m, 2H), 7.53-7.42 (m, 3H), 7.41-7.24 (m, 6H), 7.01-6.94 (m, 1H), 3.55 (s, 2H), 2.47-2.19 (m, 8H), 2.12 (s, 3H). Yield: 57%; HPLC: 99%; MS [M + 1]: 517.3 |
| 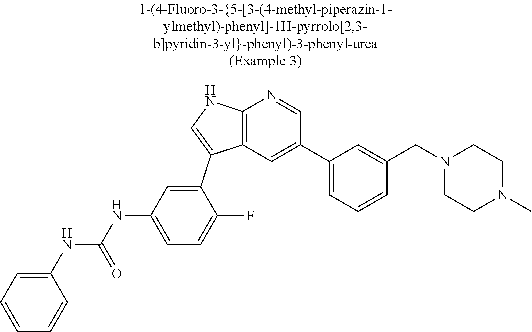 [SMILES: CN1CCN(Cc2cccc(-c3cnc4[nH]cc(-c5cc(NC(=O)Nc6ccccc6)ccc5F)c4c3)c2)CC1] | Reagents: 4-Fluoro-3-{5-[3-(4- Methyl-piperazin-1-ylmethyl)- phenyl]-1H-pyrrolo[2,3-b]pyridin-3- yl}-phenylamine and phenyl isocyanate ¹H NMR (400 MHz, DMSO-d6) δ (ppm) 12.15 (d, J = 2.1, 1H), 8.79 (bs, 1H), 8.70 (bs, 1H), 8.61 (d, J = 2.0, 1H), 8.47-8.42 (m, 1H), 8.06 (dd, J = 2.5, 6.9, 1H), 7.84 (t, J = 2.2, 1H), 7.72-7.66 (m, 1H), 7.66-7.63 (m, 1H), 7.52-7.41 (m, 3H), 7.35- 7.21 (m, 5H), 7.01-6.95 (m, 1H), 3.54 (s, 2H), 2.47-2.17 (m, 8H), 2.12 (s, 3H). Yield: 46%; HPLC: 97%; MS [M + 1]: 535.3 |

### Synthesis of Amine Compounds From Formyl-Azaindoles

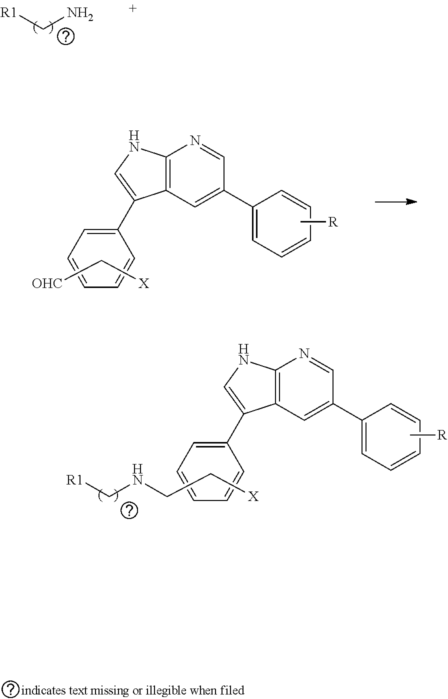

The aldehyde derivative (1 eq) and the amine derivative (1 eq) are dissolved in a methanol/acetic acid mixture (9/1). The mixture is stirred for 3 hours at room temperature and then sodium cyanoborohydride (2 eq) is added and the medium is stirred at room temperature overnight. The solvent is evaporated off, the residue is taken up in ethyl acetate, washed twice with an aqueous solution saturated with NaHCO3, once with an aqueous solution saturated with sodium chloride, dried over anhydrous Na2SO4, filtered on cotton and evaporated to dryness. The residue obtained is purified on a silica column with a gradient of 100% dichloromethane to 90/10 dichloromethane/methanol-ammonia.

| | |
|---|---|
| 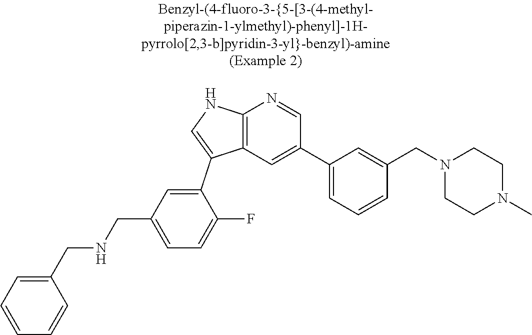 [SMILES: CN1CCN(Cc2cccc(-c3cnc4[nH]cc(-c5cc(CNCc6ccccc6)ccc5F)c4c3)c2)CC1] | Reagents: 4-Fluoro-3-{5-[3-(4-methyl- piperazin-1-ylmethyl)-phenyl]-1H- pyrrolo[2,3-b]pyridin-3-yl}- benzaldehyde and Benzylamine 1H NMR (400 MHz, DMSO-d6) δ (ppm) 12.13 (bs, 1H), 8.57 (d, J = 2.1, 1H), 8.24 (t, J = 1.9, 1H), 7.84-7.80 (m, 1H), 7.77-7.72 (m, 1H), 7.62- 7.56 (m, 2H), 7.42 (t, J = 7.6, 1H), 7.36- 7.15 (m, 8H), 3.74 (s, 2H), 3.72 (s, 2H), 3.51 (s, 2H), 2.47-2.15 (m, 8H), 2.13 (s, 3H). Yield: 25%; HPLC: 94%; MS [M + 1]: 520.3 |
| 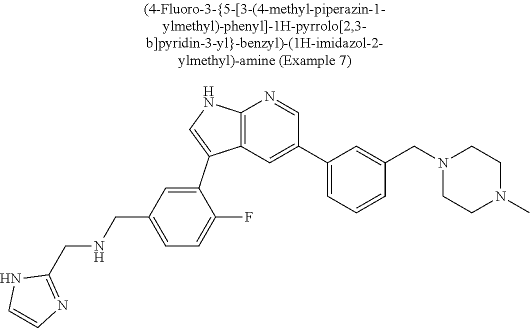 [SMILES: CN1CCN(Cc2cccc(-c3cnc4[nH]cc(-c5cc(CNCc6ncc[nH]6)ccc5F)c4c3)c2)CC1] | Reagents: 4-Fluoro-3-{5-[3-(4-methyl- piperazin-1-ylmethyl)-phenyl]-1H- pyrrolo[2,3-b]pyridin-3-yl}- benzaldehyde and (1H-Imidazol-2-yl)- methylamine 1H NMR (400 MHz, DMSO-d6) δ (ppm) 12.13 (s, 1H), 8.56 (d, J = 2.1 Hz, 1H), 8.24 (s, 1H), 7.82 (s, 1H), 7.77-7.70 (m, 1H), 7.59 (d, J = 8.8 Hz, 2H), 7.42 (t, J = 7.5 Hz, 1H), 7.29 (dd, J = 11.3, 4.7 Hz, 3H), 6.87 (s, 2H), 3.73 (d, J = 9.6 Hz, 4H), 3.52 (s, 2H), 2.47-2.20 (m, 8H), 2.14 (s, 3H). Yield: 46%; HPLC: 99%; MS [M + 1]: 510.3 |
| 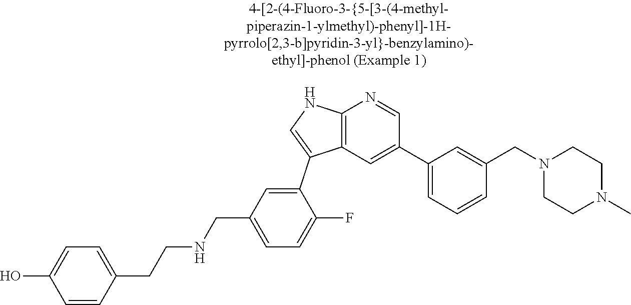 [SMILES: CN1CCN(Cc2cccc(-c3cnc4[nH]cc(-c5cc(CNCCc6ccc(O)cc6)ccc5F)c4c3)c2)CC1] | Reagents: 4-Fluoro-3-{5-[3-(4-methyl- piperazin-1-ylmethyl)-phenyl]-1H- pyrrolo[2,3-b]pyridin-3-yl}- benzaldehyde and 4-(2-Amino-ethyl)- phenol 1H NMR (400 MHz, DMSO-d6) δ (ppm) 12.23-12.01 (m, 1H), 9.08 (bs, 1H), 8.57 (d, J = 1.9, 1H), 8.27-8.19 (m, 1H), 7.85-7.78 (m, 1H), 7.76- 7.69 (m, 1H), 7.61 (s, 2H), 7.42 (t, J = 7.6, 1H), 7.33-7.20 (m, 3H), 6.95 (d, J = 8.3, 2H), 6.61 (d, J = 8.3, 2H), 3.76 (s, 2H), 3.52 (s, 2H), 2.74-2.65 (m, 2H), 2.65-2.58 (m, 2H), 2.45-2.19 (m, 8H), 2.13 (s, 3H). Yield: 25%; HPLC: 98%; MS [M + 1]: 550.3 |

Deprotection of the amino derivatives.

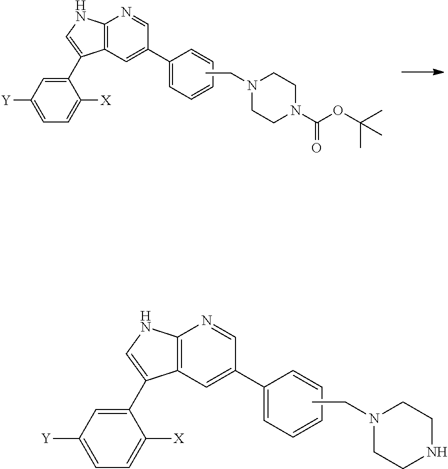

The protected amine derivative (1 eq) is dissolved in dioxane and concentrated hydrochloric acid (4 eq). The mixture is stirred for 2 hours at room temperature. The solvent is evaporated off, the residue is taken up in an ethyl acetate/water mixture. The aqueous phase is neutralized by adding NaHCO3 powder. The precipitate is filtered off and then washed with water and dried.

### Example 2. Biological Tests

A—Selectivity of the Compounds of the invention with Respect to the kinase AxL

The compounds of the invention were evaluated for their inhibitory activity on the kinase Axl.

### 1—Inhibition of the Phosphorylation of th Kinase Axl In Cellulo

The A549 cells were preincubated for 1 hour with 2.5 μM of compound 1, 2, or 3. They were then stimulated for 5 min with 1 mM pervanadate, a compound known to activate the kinase Axl by phosphorylation.

The inhibition of the phosphorylation of the Y779 residue of the intracytoplasmic domain of Axl is visualized by immunoblot using the antibody R&D Systems (AF2228).

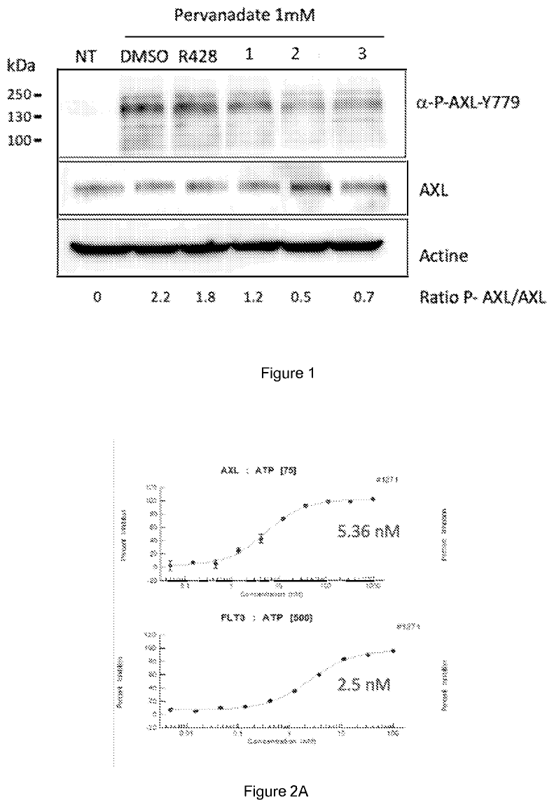

The results are shown in FIG. 1. They show a much lower or even zero activation of the Y779 residue of AxL in the presence of compounds 1, 2 and 3 compared to the control (DMSO).

### 2—Selective Inhibition of the Kinase Axl

The inhibitory activity of the compounds on a panel of kinases including, inter alia, AXL and FLT3 was evaluated by Thermo Fisher Scientific using Z′-LYTE® and Adapta® Select Screen technology.

The fluorescence-based biochemical test uses coupled enzymes and is based on the difference in sensitivity of the phosphorylated and non-phosphorylated peptides to proteolytic cleavage. The peptide substrate is labeled with 2 fluorophores—one at each end—allowing fluorescence transfer (FRET).

Compounds 1, 2 and 3 are tested in wells at the final concentration of 100 nM in 1% DMSO.

The results are presented in the following table:

| | Compound | Compound | Compound |
|---|---|---|---|
| | #1 | #2 | #3 |
| ABL1 | 38 | 16 | 22 |
| ALK | 45 | 12 | 17 |
| AURKA (Aurora A) | 14 | 11 | 14 |
| AURKB (Aurora B) | 41 | 20 | 38 |
| AXL | 95 | 95 | 94 |
| BTK | 12 | 10 | 10 |
| CSF1R (FMS) | 62 | 41 | 26 |
| CSK | 7 | 12 | 8 |
| CHUK (IKKalpha) | 43 | 14 | 4 |
| FGFR1 | 43 | 20 | 27 |
| FGFR2 | 58 | 25 | 37 |
| FLT1 (VEGFR1) | 53 | 19 | 19 |
| FLT4 (VEGFR3) | 64 | 33 | 37 |
| IKBKB (IKK beta) | 33 | 28 | 13 |
| JAK2 | 19 | 23 | 13 |
| JAK3 | 42 | 18 | 13 |
| KDR (VEGFR2) | 85 | 68 | 55 |
| KIT | 8 | 49 | 15 |
| LCK | 50 | 63 | 42 |
| MAPK1 (ERK2) | 11 | 6 | 6 |
| MERTK (cMER) | 98 | 96 | 75 |
| MET (cMet) | 15 | 92 | 16 |
| NTRK1 (TRKA) | 53 | 96 | 69 |
| NTRK2 (TRKB) | 61 | 103 | 94 |
| NUK1 (ARK5) | 74 | 16 | 14 |
| PDGFRA (PDGFR alpha) | 57 | 31 | 21 |
| PDGFRB (PDGFR beta) | 37 | 30 | 19 |
| PLK1 | 4 | 4 | 6 |
| PRKAA (PKA) | 8 | 6 | 7 |
| PTK2 (FAK) | 22 | 33 | 24 |
| RET | 84 | 48 | 23 |
| ROCK2 | 16 | 5 | 9 |
| ROS1 | 64 | 77 | 18 |
| RPS6KA1 (RSX1) | 38 | 16 | 41 |
| SRC | 19 | 16 | 10 |
| SYK | 4 | 11 | 8 |
| TEK (Tie2) | 13 | 88 | 27 |
| TYK2 | 5 | 5 | 7 |
| TYRO3 (RSE) | 51 | 67 | 19 |
| YES1 | 18 | 15 | 11 |
| ZAP70 | 4 | 4 | 5 |

Percentage inhibition of the kinases by the compounds of the invention. Columns from left to right: the kinases tested—compound 1—compound 2—compound 3.

The results show that each of the compounds of the invention shows an excellent activity (95%, 95% and 94% inhibition) on the kinase Axl when compared to other kinases, the results are disparate from one compound to another.

To calculate the IC₅₀ inhibition of Axl and FLT3 by the compounds of the invention, 10 concentrations of each compound 1, 2 and 3 were prepared by 1/3 serial dilutions from the starting concentration. The Axl or FLT3 kinases were diluted at a two-fold concentration in kinase buffer. All the ATP solutions are diluted in a 4-fold working concentration (50 mM HEPES PH 7.5, 0.01% BRIJ-35, 10 mM MgCl2, 1 mM EGTA).

Each compound is incubated at 10 concentrations between 100 nM and 0.00495 nM in order to calculate the IC₅₀.

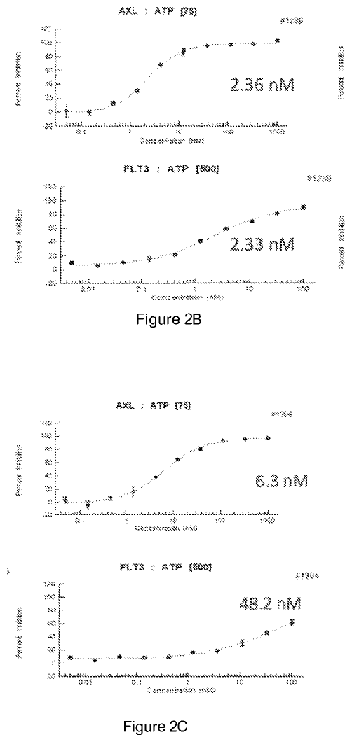

The results are shown in FIG. 2 and summarized in the table below

| | | |
|---|---|---|
| Compound | IC₅₀ Axl | IC₅₀ FLT3 |

| | | | | |
|---|---|---|---|---|
| 1 | 5.36 | nM | 2.5 | nM |
| 2 | 2.36 | nM | 2.33 | nM |
| 3 | 6.3 | nM | 48.2 | nM |

Compounds 1, 2 and 3 show an excellent activity with respect to the two kinases Axl and FLT3. Compound 3 even shows a selectivity with respect to Axl relative to FLT3, as Axl plays a critical role in the activation of FLT3.

### B—Antiviral Evaluation

The compounds of the invention were evaluated for their antiviral activity in vitro, by testing the inhibitory effect of the molecules on the replication of infectious complete viruses. The selected cell systems (HeLa, A549, A549-ACE2 cells) are relevant for the envisaged infectious models and have been validated for the expression of the Axl molecule in flow cytometry.

### Infectious Models Used:

| Virus | Model | Strain | Cells |
|---|---|---|---|
| ZIKV | mCherry | BeH-8 | HeLa |
| | reporter | | |
| DENV | Luciferase | DENV2 | HeLa |
| | reporter | | |
| WNV | Luciferase | Kunjin (KUNV) | HeLa |
| | reporter | | |
| CKV | Luciferase | LROPY-1 | HeLa |
| | reporter | | |
| SARS-COV-2 | Viral isolate | BetaCoV/France/ | A549-ACE2, |
| | | IDF0371/202 | VeroE6, primary |
| | | | bronchial |
| | | | epithelium |

### 1—Antiviral Activity Against the Zika Virus

The tests are carried out by preincubating 2.5×10⁴ cells of the HeLa line (cervical carcinoma, ATCC #CCL-2) cultured in a 96-well plate with increasing concentrations of compounds for 1 hour at 37° C. The cells are cultured in a 5% CO2 atmosphere in a Dulbecco's modified Eagle's medium (DMEM) comprising 10% calf serum and 1% penicillin/streptomycin. The cells are then exposed to the ZIKV BeH8 strain (MOI=0.1) for 1 hour in the presence of the compound.

The viral inoculum is then removed and the cells are maintained in the presence of inhibitors for 24 h. The infection of the cells is determined by quantification of the viral RNAs detected by qRT-PCR using the Luna Universal One-Step RT-PCR kit and the primers 5′-CCGCTGCCCAACACAAG and 5′-CCACTAACGTTCTTTTGCAGACAT using the Luna Universal One-Step RT-PCR kit. The values (DCT) are normalized relative to the quantification of the mRNA of the GAPDH. The values represented are averages of triplicates+standard deviation.

The control conditions were carried out by incubating the cells in the presence of DMSO, the solvent used for the solubilization of the compounds tested. The volumes of DMSO used under these conditions correspond to the volumes provided by the compounds under the test conditions.

Parallel treatment, under the same experimental conditions, was carried out with the

R428/Bemcentinib, Axl inhibitors in clinical trial phase developed by the company Bergenbio.

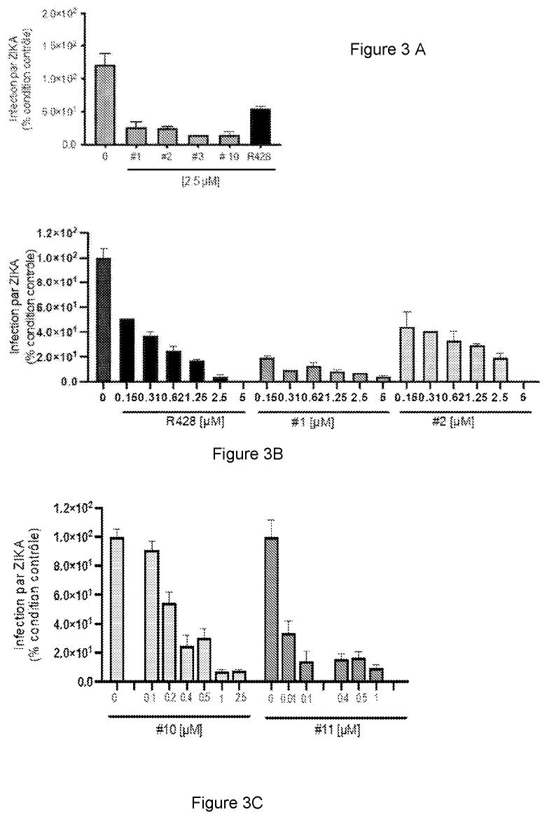

The results obtained with compounds 1, 2, 3, 10 and 11 are shown in FIG. 3. The IC₅₀ of compounds 1, 10 and R428/Bemceinib were determined using the software Graphpad Prism.

Under these experimental conditions, the compounds 1, 2, 3, 10, and 11 used at a concentration greater than or equal to 150 nM prevent infection of the Hela cells by the strain ZIKV BeH-8. The in vitro efficacy of these compounds is at least equivalent to that of R428/Bemcentinib described in the literature for its efficacy against the ZIKV virus at concentrations of 1 to 3 μM.

### 2—Antiviral Activity Against the Dengue Virus

The tests are carried out by preincubating 4×10⁴ cells of the HeLa line (adenocarcinoma, Human Cervical; ATCC #CCL-2) cultured in a 96-well plate with increasing concentrations of compounds for 1 hour. The cells are cultivated in a 5% CO2 atmosphere in a Dulbecco's modified Eagle's medium (DMEM) comprising 10% calf serum and 1% penicillin/streptomycin. The cells are then exposed to the DENV subtype 2 strain (DENV2) expressing a nanoluciferase gene upstream of the sequence coding for the capsid protein, in the presence of the compounds, for 1 hour. The viral inoculum is then removed and the cells are maintained in the presence of the inhibitors for 3 days.

The cells are lysed using Passive lysis Buffer (Promega) reagent. The viral infection is detected by quantification of the nanoluciferase activity in the cell lysate in the presence of the reagent Genofax A (Yelen) and using an Infinite F200PRO (Tecan) fluorimeter. The values are normalized relative to the amount of total proteins contained in the cell lysate and determined using the BCA Protein Assay kit (Pierce) by measuring the absorbance at 562 nm. The values represented are averages of triplicates+standard deviation. The control conditions are identical to those described above.

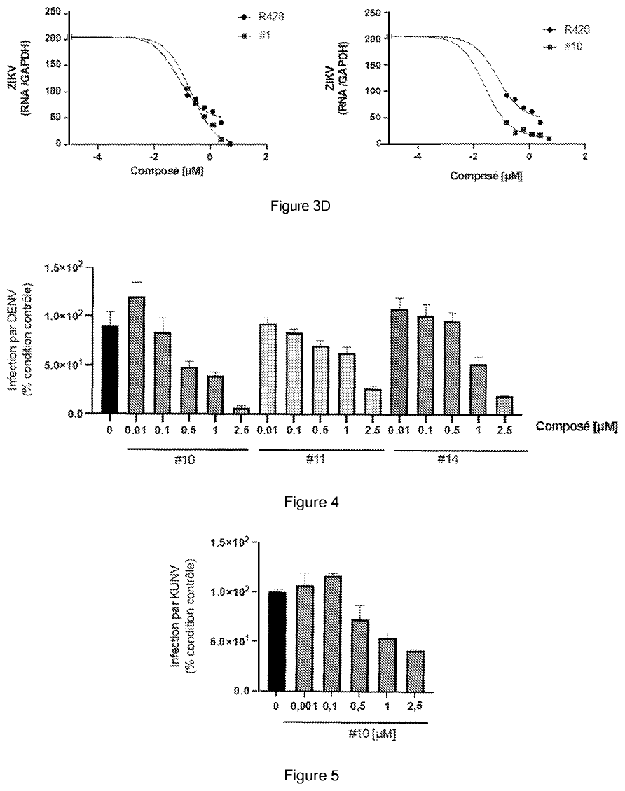

The results obtained for compounds 10, 11 and 14 are shown in FIG. 4.

Under these experimental conditions, compounds 10, 11 and 14 used at a concentration greater than or equal to 500 nM prevent infection of Hela cells by subtype DENV2.

### 3—Antiviral Activity Against the WNV Virus, Kunjin Strain

The tests are carried out by preincubating 4×10⁴ cells of the HeLa line (ATCC #CCL-2) cultured in a 96-well plate with increasing concentrations of compounds for 1 hour. The cells are cultivated in a 5% CO2 atmosphere in a Dulbecco's modified Eagle's medium (DMEM) comprising 10% calf serum and 1% penicillin/streptomycin. The cells are then exposed to the WNV virus, Kunjin strain expressing a nanoluciferase gene upstream of the sequence coding for the capsid protein, in the presence of the compounds, for 1 hour. The viral inoculum is then removed and the cells are maintained in the presence of the inhibitors for 24 hours.

The cells are lysed using Passive lysis Buffer (Promega) reagent. The viral infection is detected by quantification of the nanoluciferase activity in the cell lysate in the presence of the reagent Genofax A (Yelen) and using an Infinite F200PRO (Tecan) fluorimeter. The values are normalized relative to the amount of total proteins contained in the cell lysate and determined using the BCA Protein Assay kit (Pierce) by measuring the absorbance at 562 nm. The values represented are averages of triplicates +standard deviation. The control conditions are identical to those described above.

The results obtained for compound 10 are shown in FIG. 5.

Under these experimental conditions, compound 10 used at a concentration greater than or equal to 500 nM prevents infection of Hela cells with WNV strain Kunjin.

### 4—Antiviral Activity Against the Chikungunya Virus

The tests are carried out by preincubating 4×10⁴ cells of the HeLa line (ATCC #CCL-2) cultured in a 96-well plate with increasing concentrations of compounds for 1 hour. The cells are cultivated in a 5% CO2 atmosphere in a Dulbecco's modified Eagle's medium (DMEM) comprising 10% calf serum and 1% penicillin/streptomycin. The cells are then exposed to the CHIKV virus, LR-OPY1 strain expressing either a luciferase gene, or a sequence encoding GFP upstream of the sequence coding for the nsP3 protein. The cells are incubated for 1 hour in the presence of the compounds.

The viral inoculum is then removed and the cells are maintained in the presence of inhibitors for 24 h. The viral infection is measured after lysis of the cells using the Passive lysis Buffer (Promega) reagent, either by direct quantification of the fluorescence of GFP or by quantification of the firefferase activity in the lysate of the cells carried out in the presence of the Genofax A (Yelen) reagent and using an Infinite F200PRO (Tecan) fluorimeter. The values are normalized relative to the amount of total proteins contained in the cell lysate and determined using the BCA Protein Assay kit (Pierce) by measuring the absorbance at 562 nm. In both protocols, the values are normalized relative to the amount of total proteins contained in the cell lysate and determined using the BCA Protein Assay kit (Pierce). The values represented are averages of triplicates+standard deviation. The control conditions are identical to those described above.

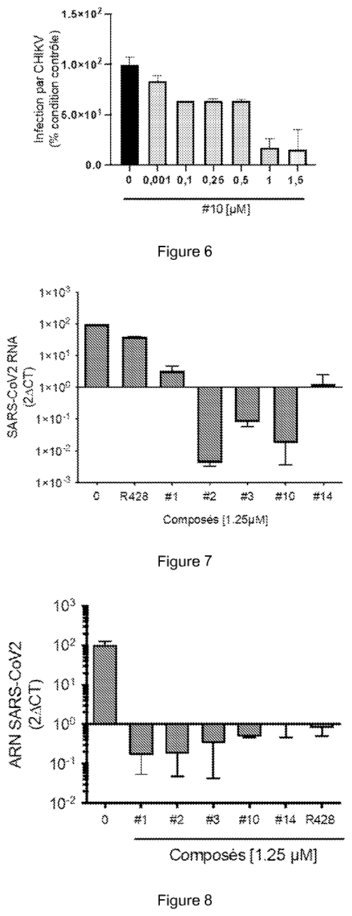

The results obtained for compound 10 are shown in FIG. 6.

Under these experimental conditions, the compound 10 used at a concentration greater than or equal to 100 nM prevents infection of the Hela cells by the Chikungunya virus.

For each of the tested infectious models, the IC₅₀ active compounds were determined using the software Graphpad Prism.

| | Virus | | | | | | | |
|---|---|---|---|---|---|---|---|---|
| Compound | CKV | | KUNV | | DENV | | ZIKV | |
| 1 | | | | | | | 198 | Nm |
| 2 | | | | | | | 25 | nM |
| 10 | 572 | nM | 836 | nM | 626 | nM | 397 | nM |
| 11 | | | | | >1 | μM | <100 | nM |
| 14 | | | >1 | μM | >1 | μM | | |
| R428/Bemcentinib | 239 | nM | ND | | 488 | nM | 285 | nM |
| (reference) | | | | | | | | |

These evaluations indicate that several compounds have an EC₅₀ less than 1 μM.

### 5—Antiviral Activity Against the Coronavirus SARS-COV-2

The inhibitory activity of the compounds of the invention on the infection by SARS-COV-2 was evaluated in vitro by infection of three relevant lines for the study of this pathogen: the green monkey line VeroE6 (immortalized kidney epithelium, ATCC #CRL-1586), the A549-ACE2 line produced by transfection of the ACE2 receptor in the A549 line (pulmonary epithelial carcinoma; ATCC #CCL-185), commercial primary bronchial epithelium cells (Mucilair Epithelix).

a—Assessment of Antiviral Activities in VeroE6 and A549-ACE2 Cells

The infection tests are carried out by preincubating 4×10⁴ VeroE6 or A549-ACE2 cells, cultivated in a 96-well plate, with the indicated concentrations of compounds for 1 hour. The cells are cultivated in a 5% CO2 atmosphere in a Dulbecco's modified Eagle's medium (DMEM) comprising 2% calf serum and 1% penicillin/streptomycin. The cells were then exposed to the SARS-COV-2 virus, BetaCoV/France/IDF0371/2020 (MOI=0.01) for 2 hours in the presence of the compounds before elimination of the innoculum, washing and culturing the cells in the presence of the compounds. 24 h after the viral challenge, the infection is quantified by amplification of the viral RNA by qRT-PCR using the Luna Universal One-Step RT-PCR kit. The values (DCT) are normalized by the GAPDH mRNA. The values represented are averages of triplicates+standard deviation. The control conditions were carried out by incubating the cells in the presence of DMSO, the solvent used for the solubilization of the compounds tested. The volumes of DMSO used under these conditions correspond to the volumes provided by the compounds under the test conditions.

Parallel treatment, under the same experimental conditions, was carried out with the R428/Bemcentinib, Axl inhibitors in clinical trial phase developed by the company Bergenbio.

The results obtained for compounds 1, 2, 3, 10 and 14 are illustrated in FIG. 7 (VeroE6 line) and 8 (A549-ACE2 line).

Under these experimental conditions, compounds 1, 2, 3, 10 and 14 prevent the infection of the VeroE6 and A549-ACE2 cells by the SARS-COV-2 virus.

IC₅₀ compounds 1, 2 and 3 were determined on the VeroE6 and A549-ACE2 cells using the software Graphpad Prism.

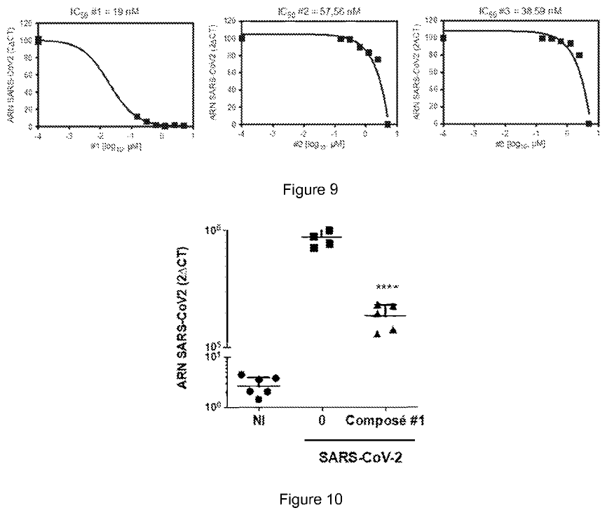

The results obtained are shown in FIG. 9 and in the following table:

| | IC₅₀ | | |
|---|---|---|---|
| Compound | 1 | 2 | 3 |
| A549-ACE2 | 19 nM | 57 nM | 38 nM |
| Vero E6 | 77 nM | 289 nM | ND |

### Compounds 1, 2, 3, 10 and 14 Exhibit an Anti-SARS-COV-2 Activity In Vitro.

b—Assessment of Antiviral Activities in a Human Epithelium Model Antiviral

The infection tests of the primary bronchial epithelium were carried out from the model

MucilAir™ Human airway epthelial cells (Epithelix) cultured in a suitable medium (HAE medium, Epithelix). The compounds are added to the basal compartment of the culture and preincubated for 1 hour at 37° C. The viral inoculum (MOI=0.01) is then added to the apical compartment at the same time as the compounds. After 2 hours at 37° C., the viral inoculum is removed, replaced by medium containing the test compound and the culture is kept at 37ºC for 24 hours. The infection of the cells is determined by quantification of the viral RNA detected by qRT-PCR using the Luna Universal One-Step RT-PCR kit. The values (DCT) are normalized with respect to the GAPDH mRNA.

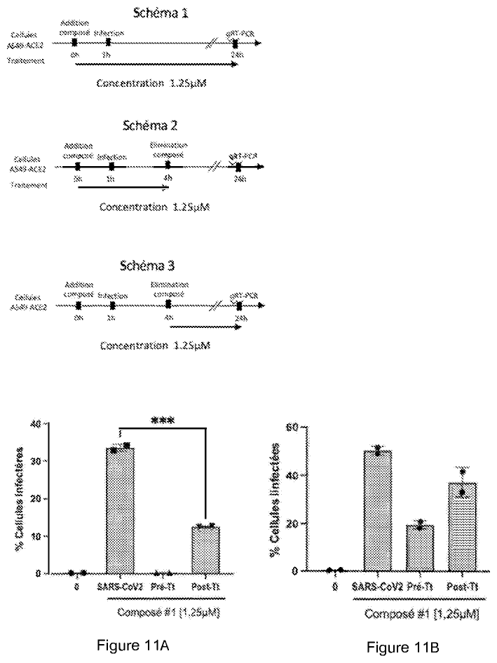

The results obtained for compound 1 are shown in FIG. 10.

These assessments indicate that compound 1 inhibits the infection of the primary bronchial epithelium by SARS-COV-2.7. Preventive and curative activity of compound #1 against SARS-COV-2 coronavirus

The inhibitory activity of compound 1 on infection by SARS-COV-2 was assessed in vitro in the context of a preventive treatment (Pre-Tt) or curative treatment (Post-Tt) of the A549-ACE2 line produced by transfection of the ACE2 receptor in the A549 line (pulmonary epithelial carcinoma; ATCC # CCL-185) a), and of the green monkey line VeroE6 (immortalized kidney epithelium, ATCC #CRL-1586).

The infection tests are carried out on 4×10⁴ cells cultured in a 96-well plate, in a 5% CO₂ atmosphere in a Dulbecco's modified Eagle's medium (DMEM) comprising 2% calf serum and 1% penicillin/streptomycin. The cells are exposed to the SARS-COV-2 virus, BetaCoV/France/IDF0371/2020 (MOI=0.01). The compounds are added to the cells i.e. 1 hour before infection and maintained for 24 hours; or added to the cells 1 hour before the infection and removed by washing 4 hours after the viral challenge; is added to the cells 4 hours after exposure to the virus and maintained for 24 hours (Post-Tt). The residual infection is quantified by amplification of the viral RNA by qRT-PCR using the Luna Universal One-Step RT-PCR kit. The values (DCT) are normalized with respect to the GAPDH mRNA.

The control conditions were carried out by incubating the cells in the presence of DMSO (SARS-COV-2), the solvent used for the solubilization of the compounds tested. The volumes of DMSO used under these conditions correspond to the volumes provided by the compounds under the test conditions.

The results obtained are shown in FIG. 11.

These evaluations indicate that the preventive use of the compounds inhibits the infection by SARS-COV-2 in human A549-ACE2 cells and VeroE6 monkey cells. The administration of the compounds after the viral challenge reduces the viral multiplication in the A549-ACE2 cells, but not in the VeroE6 line, which is characterized by a defect in producing type I interferons.

In conclusion, the active compounds on the various infectious models tested are as follows:

| | Virus | | | | | |
|---|---|---|---|---|---|---|
| | | | | | | SARS- |
| Compound | CHIKV | MAYV | KUNV | DENV | ZIKV | COV-2 |
| 1 | ND | ND | ACTIVE | ND | ACTIVE | ACTIVE |
| 2 | ND | ND | ACTIVE | ND | ACTIVE | ACTIVE |
| 3 | ND | ND | ND | ND | ND | ACTIVE |
| 10 | ACTIVE | ACTIVE | ACTIVE | ACTIVE | ACTIVE | ACTIVE |
| 11 | ND | ND | ND | ACTIVE | ACTIVE | ND |
| 14 | ND | ND | ACTIVE | ACTIVE | ND | ND |

1. A compound of formula (I): 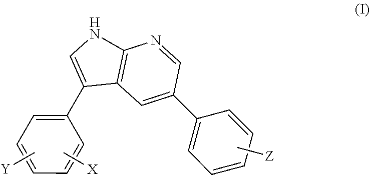
in which
X represents a hydrogen atom or a halogen atom,
Y is selected from a hydrogen atom, a —CN, —CH₂OH, —CH═NOH, —CO₂H group, an aryl optionally mono- or polysubstituted, a heteroaryl optionally mono- or polysubstituted, and -L-(CH₂)p-W,
L represents —C(O)NH—, —CH₂NH—, —NHC(O)—, —CH₂N═CH—, —CH═N—, —NHC(O)NH— or —CH₂NHC(O)—,
p is an integer from 0 to 2,
when L represents —CH₂NH—, —NHC(O)—, —CH₂N═CH—, —CH═N—, —NHC(O)NH— or —CH₂NHC(O)—, W is selected from:
a hydrogen atom,
a C₁-C₆ alkyl, preferably a methyl,
a C₆-C₁₀ aryl, optionally mono- or polysubstituted, advantageously by 1 to 3 groups independently selected from a halogen atom, a hydroxyl, a C₁-C₆ alkyl, or a C1-C₆ alkoxyl, and
a 5- to 10-membered heteroaryl containing from 1 to 3 heteroatoms independently selected from N, O and S, said heteroaryl optionally being mono- or polysubstituted, advantageously it is optionally substituted with 1 to 4 groups independently selected from
a halogen atom,
a hydroxyl,
an oxo group,
a C₁-C₆ alkoxyl,
a C₁-C₆ alkyl, wherein 1 or several hydrogen atoms is optionally replaced with a fluorine atom, and
a C₆-C₁₀ aryl, optionally substituted with a halogen atom and/or a C₁-C₆ alkyl;
when L represents -C(O)NH-, W is selected from:
an optionally mono- or polysubstituted phenyl, advantageously it is optionally substituted with a halogen atom, a hydroxyl group, a C₁-C₆ alkyl, or a C₁-C₆ alkoxyl group, and
a 5- to 10-membered heteroaryl comprising from 1 to 2 heteroatoms independently selected from N, O and S, said heteroaryl optionally being mono- or polysubstituted, advantageously it is optionally substituted with 1 to 4 substituents independently selected from
a halogen atom,
a hydroxyl,
an oxo group,
a C₁-C₆ alkoxyl group,
a C₁-C₆ alkyl, wherein 1 or several hydrogen atoms is optionally replaced with a fluorine atom, and
a C₆-C₁₀ aryl, optionally substituted with a halogen atom and/or a C₁-C₆ alkyl;
Z represents —CH₂Q or —O(CH₂)ₘT,
Q represents —OH, —SO₂R₁, —NR₂R₃, or —R₄
R₁ represents a C₁-C₆ alkyl,
R₂ and R₃ each independently represent a C₁-C₆ alkyl, and
R₄ represents a 5-7 membered heterocycloalkyl, comprising 1 to 2 heteroatoms independently selected from N, O and S, said heterocycloalkyl optionally substituted with 1 to 3 substituents independently selected from a C₁-C₆ alkyl, a C₁-C₆ C(O)-alkyl and a C₁-C₆ C(O)O-alkyl,
m is equal to 1 or 2,
T is —O(CH₂)ₙCH₃ or —R₅
n is 0 or 1
R₅ is a 5-7 membered heterocycloalkyl, comprising 1 to 2 heteroatoms independently selected from N, O and S, said heterocycloalkyl optionally substituted with 1 to 3 substituents independently selected from a C₁-C₆ alkyl, a C₁-C₆ C(O)-alkyl and a C₁-C₆ C(O)O-alkyl;
or a pharmaceutically acceptable salt thereof or a mixture thereof,
for use in the prevention and/or treatment of viral infections.

2. The compound for its use according to claim 1, of formula (Id): 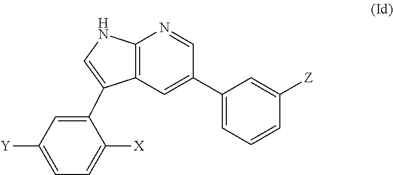
wherein X, Y and Z are as defined in claim 1.

3. The compound for its use according to claim 1, characterized in that Z represents —CH₂R₄ wherein R₄ represents a piperazine group, optionally substituted with a C₁-C_{6,} alkyl, in particular a methyl.

4. The compound for its use according to claim 1, characterized in that X represents a halogen, in particular fluorine.

5. The compound for its use according to claim 1, characterized in that Y represents L-(CH₂)ₚ-W, L representing -CH₂NH or —NHC(O)NH—, p being an integer from 0 to 2, and W being selected from:
a phenyl optionally substituted with a hydroxyl group, or a C₁-C₆ alkyl group, and
a 5- to 10-membered heteroaryl comprising from 1 to 2 heteroatoms independently selected from N, O or S.

6. The compound for its use according to claim 5, characterized in that Y represents L-(CH₂)ₚ-W, L representing —CH₂NH or —NHC(O)NH—, p being an integer from 0 to 2, and W being selected from:
a phenyl optionally substituted with a hydroxyl group, and
an imidazole.

7. The compound for its use according to claim 1, characterized in that Y represents —NHC(O)-W, with W representing a 5- to 10-membered heteroaryl group, comprising from 1 to 2 heteroatoms independently selected from N, O or S, said heteroaryl being optionally substituted with 1 to 4 substituents independently selected from:
an oxo group,
a C₁-C₆ alkyl group, wherein 1 or several hydrogen atoms is optionally replaced with a fluorine atom, and
a C₆-C₁₀ aryl group, optionally substituted with a halogen atom and/or a C₁-C₆ alkyl group.

8. The compound for its use according to claim 7, characterized in that W is 2(1H)-pyridinone, optionally substituted with 1 to 4 substituents independently selected from:
a methyl, and
a phenyl optionally substituted with a fluorine atom.

9. A compound for its use according to claim 1, selected from: 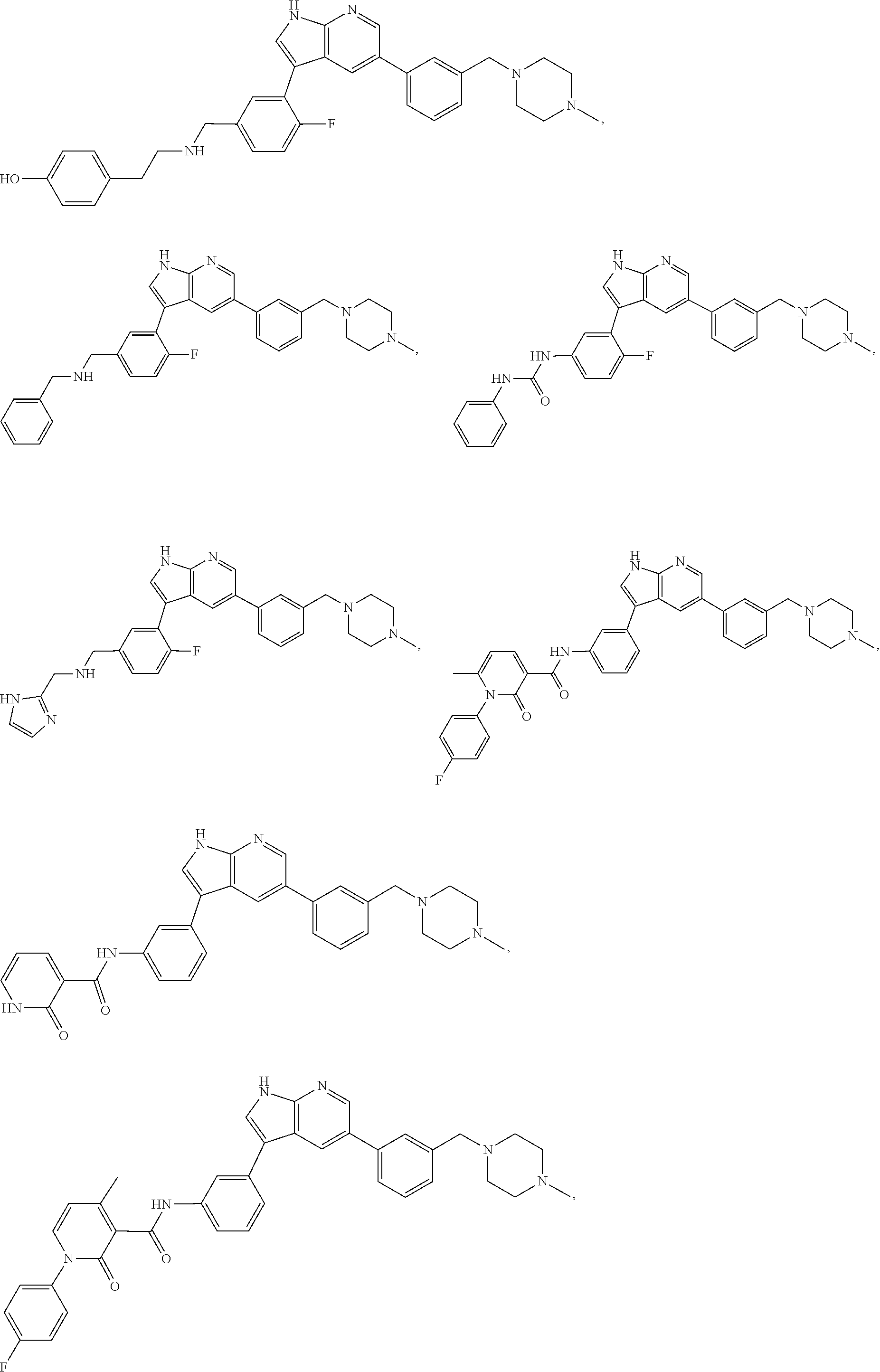 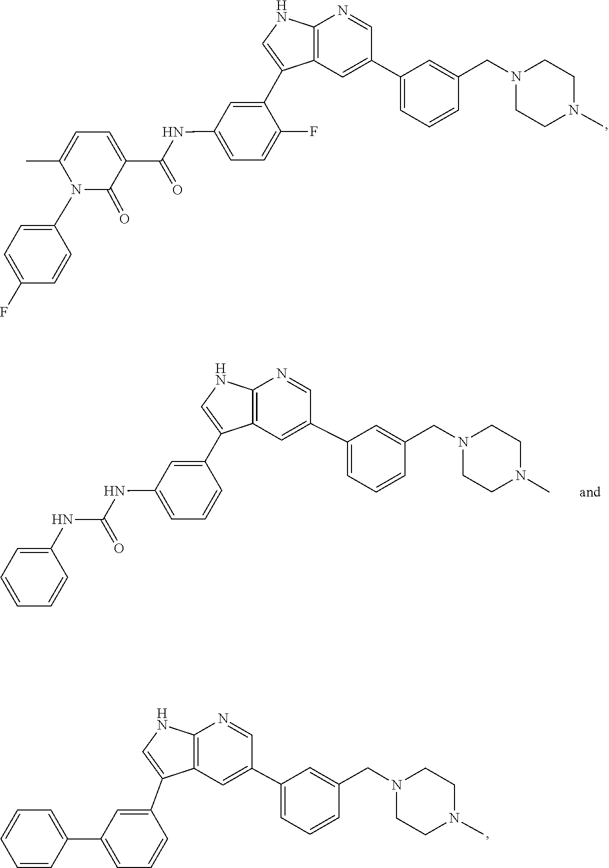 [SMILES: Cc1ccc(C(=O)Nc2ccc(F)c(-c3c[nH]c4ncc(-c5cccc(CN6CCN(C)CC6)c5)cc34)c2)c(=O)n1-c1ccc(F)cc1 ; CN1CCN(Cc2cccc(-c3cnc4[nH]cc(-c5cccc(NC(=O)Nc6ccccc6)c5)c4c3)c2)CC1 ; CN1CCN(Cc2cccc(-c3cnc4[nH]cc(-c5cccc(-c6ccccc6)c5)c4c3)c2)CC1]
preferably from

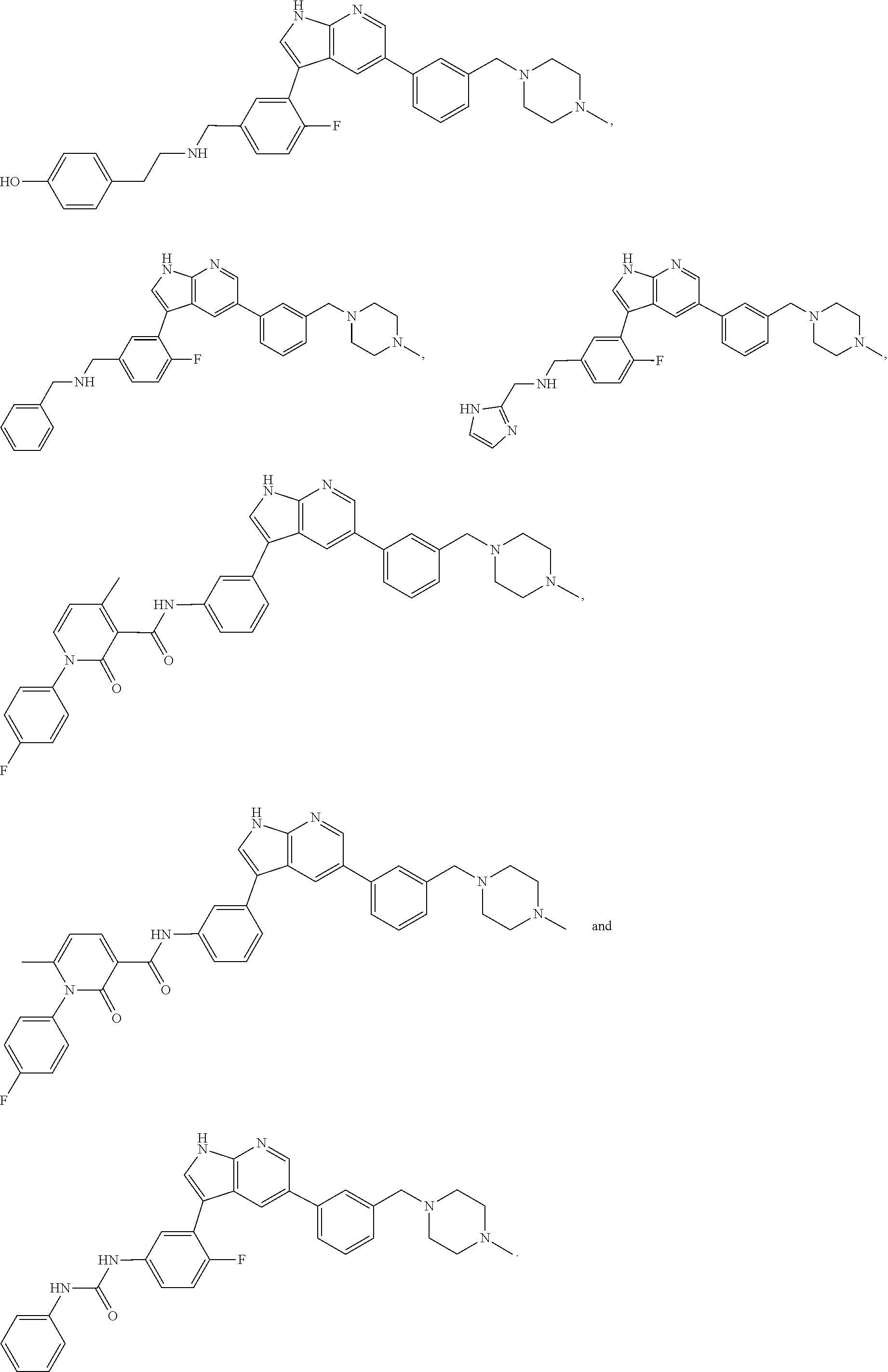 [SMILES: CN1CCN(Cc2cccc(-c3cnc4[nH]cc(-c5cc(CNCCc6ccc(O)cc6)ccc5F)c4c3)c2)CC1 ; CN1CCN(Cc2cccc(-c3cnc4[nH]cc(-c5cc(CNCc6ccccc6)ccc5F)c4c3)c2)CC1 ; CN1CCN(Cc2cccc(-c3cnc4[nH]cc(-c5cc(CNCc6ncc[nH]6)ccc5F)c4c3)c2)CC1 ; Cc1ccn(-c2ccc(F)cc2)c(=O)c1C(=O)Nc1cccc(-c2c[nH]c3ncc(-c4cccc(CN5CCN(C)CC5)c4)cc23)c1 ; Cc1ccc(C(=O)Nc2cccc(-c3c[nH]c4ncc(-c5cccc(CN6CCN(C)CC6)c5)cc34)c2)c(=O)n1-c1ccc(F)cc1 ; CN1CCN(Cc2cccc(-c3cnc4[nH]cc(-c5cc(NC(=O)Nc6ccccc6)ccc5F)c4c3)c2)CC1]

10. A pharmaceutical composition comprising a compound according to claim 1 or a pharmaceutically acceptable salt thereof as an active ingredient, and a pharmaceutically acceptable excipient for use in the prevention and/or treatment of viral infections.

11. The pharmaceutical composition for its use according to claim 10, further comprising an additional therapeutic agent, chosen from an antiviral agent, an anti-inflammatory agent, an immunomodulatory or immunosuppressive agent, an agent for the treatment of immunodeficiency disorders and an agent for pain treatment.

12. A compound for its use according to claim 1, wherein or whereby the viral infections are due to Flaviviruses, such as dengue virus, Zika virus, West Nile virus, Kunjin virus, Alphaviruses, such as Chikungunya virus, Mayaro virus, Semliki Forest virus, Sindbis virus, Eastern equine encephalitis virus, Western equine encephalitis virus, Venezuelan equine encephalitis virus, Filoviruses such as Ebola virus, Marburg fever virus, arenaviruses such as Lassa fever virus, Junin virus, Amapari virus, picornaviruses such as vesicular stomatitis virus, paramyxoviruses such as influenza virus, or coronaviruses such as SARS-COV-2.
